(19) United States
(12) Patent Application Publication
(10) Pub. No.: US 2024/0117336 A1
(43) Pub. Date: Apr. 11, 2024

(54) METHODS AND COMPOSITIONS FOR DNA BASED KINSHIP ANALYSIS

(71) Applicant: Verogen, Inc., San Diego, CA (US)

(72) Inventors: Cydne L. HOLT, Encinitas, CA (US); Paulina WALICHIEWICZ, San Diego, CA (US); Elmira FOROUZMAND, San Diego, CA (US); Kathryn M. STEPHENS, Poway, CA (US); Seth STADICK, Boulder, CO (US); Tim FENNELL, Boulder, CO (US)

(73) Assignee: Verogen, Inc., San Diego, CA (US)

(21) Appl. No.: 18/276,845

(22) PCT Filed: Feb. 10, 2022
(86) PCT No.: PCT/US2022/015944

§ 371 Date: Aug. 10, 2023

Related U.S. Application Data
(60) Provisional application No. 63/149,071, filed on Feb. 12, 2021.

(51) Int. Cl. C12N 15/10; C12Q 1/6858; C12Q 1/6876

(52) U.S. Cl. CPC C12N 15/1065; C12Q 1/6858; C12Q 1/6876; C12Q 2600/156; C12Q 2600/16

(57) ABSTRACT

The present disclosure in some aspects relates to performing DNA based kinship analysis involving analysis of between 5,000 and 50,000 SNPs, including sample preparation and sequencing technologies and methods that can be used to calculate the degree of relationship of a DNA profile to one or more reference DNA profiles.

### CROSS-REFERENCE TO RELATED APPLICATIONS

This application claims priority from U.S. provisional application No. 63/149,071 filed Feb. 12, 2021, entitled “Methods and Compositions for DNA Based Kinship Analysis,” the contents of which are hereby incorporated by reference in their entirety.

### FIELD

The present disclosure relates in some aspects to methods and compositions for DNA based kinship analysis in a sample.

### BACKGROUND

Current methods of generating DNA profiles for comparisons in genetic databases include genotyping using dense SNP microarrays and whole genome sequencing (WGS) followed by association of evidentiary samples with distant relatives in databases, which require high quantity and high quality DNA samples, and are not designed for familial searching or forensic purposes. Forensic casework samples are generally low quantity and low quality samples, and data from the current methods requires extensive imputation to generate results capable of being uploaded to a search database. Therefore, there is need for a new and improved method for the generation of DNA based profile analysis.

### SUMMARY

Provided herein in some aspects are methods, compositions, devices, and systems for using forensically curated Single Nucleotide Polymorphism (SNP) marker sets to provide comprehensive, accessible and complete DNA based analysis profiles.

Provided herein is a method for performing DNA-based kinship analysis, comprising: providing a nucleic acid sample, amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions, generating a nucleic acid library from the amplification products, sequencing the nucleic acid library generated from the amplification products, analyzing the sequences of the amplification products, determining the genotypes of the plurality of SNPs, thereby generating a DNA profile, and calculating the degree of relationship of the DNA profile to one or more reference DNA profiles.

Also provided herein is a method for performing DNA-based kinship analysis, comprising: providing a nucleic acid sample, amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions, generating a nucleic acid library from the amplification products sequencing the nucleic acid library generated from the amplification products, determining the genotypes of the plurality of SNPs, thereby generating a DNA profile, and calculating the degree of relationship of the DNA profile to one or more reference DNA profiles.

In some of any of such embodiments, the sequencing is conducted using massively parallel sequencing (MPS). In some of any of such embodiments, the sequencing does not comprise whole genome sequencing (WGS).

In some of any of such embodiments, the method further comprises generating a family tree comprising the DNA profile in relation to one or more DNA profiles.

Also provided herein is a method of constructing a nucleic acid library, comprising: providing a nucleic acid sample, amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating a nucleic acid library comprising amplification products, wherein the amplification is carried out in one or more multiplex PCR reactions.

In some of any of such embodiments, the nucleic acid sample comprises genomic DNA. In some of any of such embodiments, the nucleic acid sample comprises one or more enzyme inhibitors. In some of any of such embodiments, the one or more enzyme inhibitors comprise one or more inhibitors selected from the group consisting of hematin, heme, humic acid, indigo, and tannic acid.

In some of any of such embodiments, the nucleic acid sample comprises low-quality nucleic acid molecules and/or low quantity nucleic acid molecules. In some of any of such embodiments, the low quality nucleic acid molecules are degraded genomic DNA and/or fragmented genomic DNA. In some of any of such embodiments, the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200. In some of any such embodiments, the low quality nucleic acid molecules have a DI of at least 1 and up to or less than 158.3.

In some of any of such embodiments, the nucleic acid sample comprises high quality nucleic acid molecules. In some of any of such embodiments, the high quality nucleic acid molecules have a DI of less than 1.

In some of any of such embodiments, the nucleic acid sample is a forensic sample. In some of any of such embodiments, the nucleic acid sample is derived from saliva, blood, semen, hair, teeth, or bone. In some of any of such embodiments, the nucleic acid sample is derived from saliva, blood, or semen. In some of any of such embodiments, the nucleic acid sample is derived from a buccal swab, paper, fabric, or other substrate that is impregnated with saliva, blood, semen, or other bodily fluid.

In some of any of such embodiments, the nucleic acid sample comprises between or between about 50 μg and 100 ng of genomic DNA. In some of any of such embodiments, the nucleic acid sample comprises between or between about 100 pg and 5 ng of genomic DNA or between or between about 50 pg and 5 ng of genomic DNA. In some of any of such embodiments, the nucleic acid sample comprises at or about 1 ng of genomic DNA.

In some of any of such embodiments, the plurality of SNPs comprises kinship SNPs (kiSNPs). In some of any of such embodiments, the plurality of SNPs comprises kiSNPs, biogeographical ancestry SNPs (aiSNPs), identity SNPs (iiSNPs), phenotype SNPs (piSNPs), x-chromosome SNPs (xSNPs), and y-chromosome SNPs (ySNPs). In some of any of such embodiments, the plurality of SNPs comprises SNPs selected from one or more of the groups consisting of kiSNPs, aiSNPs, iiSNPs, piSNPs, xSNPs, and ySNPs. In some of any of such embodiments, at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

Also provided herein is a method for calculating degree of relatedness, comprising: obtaining a DNA profile comprising genotypes of at least between at or about 5,000 to 50,000 SNPs; and calculating the degree of relationship of the DNA profile to one or more reference DNA profiles.

Also provided herein is a method for calculating degree of relatedness, comprising: generating a DNA profile comprising genotypes of at least between at or about 5,000 to 50,000 SNPs; and calculating the degree of relationship of the DNA profile to one or more reference DNA profiles.

In some of any of such embodiments, the calculating the degree of relationship comprises a large cohort method comprising the steps of: (1) performing a KING-Robust kinship estimation between all pairs of a sample set comprising the one or more reference DNA profiles, wherein parings with a kinship coefficient>0.01 are identified as related and parings with a kinship coefficient<−0.025 are identified as ancestry-diverged; (2) removing all reference DNA profiles that have>5% missing data; (3) rank all reference DNA profiles by identifying each reference DNA profile with a ranking value, wherein ranking value is determined based on the number of related reference DNA profiles in the full set of reference DNA profiles that is ranked from least to most and ties are broken by the number of ancestry-diverged reference DNA profiles in the full set of reference DNA profiles as ranked from most to least; and iteratively through the ranked reference DNA profiles, for each reference DNA profile: (i) if the reference DNA profile is not yet in a related sample set, add it to an unrelated sample set and add all related reference DNA profiles to the related sample set, and (ii) if the reference DNA profile is already in the related sample set, then skip to the next reference DNA profile, and repeat beginning at step (3)(i).

In some of any of such embodiments, the one or more reference DNA profiles comprises at or about or at least or at least about 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles. In some of any of such embodiments, the one or more reference DNA profiles comprises at or about or at least or at least about 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles. Also provided herein is a nucleic acid library constructed using any of the methods described herein.

Also provided herein is a plurality of primers that specifically hybridize to a plurality of target sequences comprising at least between at or about 5,000 to 50,000 single nucleotide polymorphisms (SNPs) in a nucleic acid sample, wherein amplifying the nucleic acid sample using the plurality of primers in one or more multiplex PCR reactions results in amplification products. In some embodiments, the nucleic acid sample comprises genomic DNA.

In some of any of such embodiments, the nucleic acid sample comprises one or more enzyme inhibitors. In some of any of such embodiments, the one or more enzyme inhibitors comprise one or more inhibitors selected from the group consisting of hematin, heme, humic acid, indigo, and tannic acid.

In some of any of such embodiments, the nucleic acid sample comprises low-quality nucleic acid molecules and/or low quantity nucleic acid molecules. In some of any of such embodiments, the low quality nucleic acid molecules are degraded genomic DNA and/or fragmented genomic DNA. In some of any of such embodiments, the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200. In some of any of such embodiments, the low quality nucleic acid molecules have a DI of at least 1 and up to or less than 158.3.

In some of any of such embodiments, the nucleic acid sample comprises high quality nucleic acid molecules. In some of any of such embodiments, the high quality nucleic acid molecules have a DI of less than 1.

In some of any of such embodiments, the nucleic acid sample is a forensic sample. In some of any of such embodiments, the nucleic acid sample is derived from a buccal swab, paper, fabric, or other substrate that is impregnated with saliva, blood, or other bodily fluid. In some of any of such embodiments, the nucleic acid sample comprises between or between about 50 μg and 100 ng of genomic DNA. In some of any of such embodiments, the nucleic acid sample comprises between or between about 100 pg and 5 ng of genomic DNA or between or between about 50 pg and 5 ng of genomic DNA. In some of any of such embodiments, the nucleic acid sample comprises at or about 1 ng of genomic DNA.

In some of any of such embodiments, the plurality of SNPs comprises kinship SNPs (kiSNPs). In some of any of such embodiments, the plurality of SNPs comprises kiSNPs, biogeographical ancestry SNPs (aiSNPs), identity SNPs (iiSNPs), phenotype SNPs (piSNPs), x-chromosome SNPs (xSNPs), and y-chromosome SNPs (ySNPs). In some of any of such embodiments, the plurality of SNPs comprises SNPs selected from one or more of the groups consisting of kiSNPs, aiSNPs, iiSNPs, piSNPs, xSNPs, and ySNPs. In some of any of such embodiments, at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

Also provided herein is a method for constructing a DNA profile, comprising: providing a nucleic acid sample, amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions, sequencing the amplification products, determining the genotypes of the plurality of SNPs, thereby generating a DNA profile.

In some of any of such embodiments, the sequencing does not comprise whole genome sequencing (WGS). In some of any of such embodiments, the nucleic acid sample comprises genomic DNA.

In some of any of such embodiments, the nucleic acid sample comprises one or more enzyme inhibitors. In some of any of such embodiments, the one or more enzyme inhibitors comprise one or more inhibitors selected from the group consisting of hematin, heme, humic acid, indigo, and tannic acid.

In some of any of such embodiments, the nucleic acid sample comprises low-quality nucleic acid molecules and/or low quantity nucleic acid molecules. In some of any of such embodiments, the low quality nucleic acid molecules are degraded genomic DNA and/or fragmented genomic DNA. In some of any of such embodiments, the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200. In some of any of such embodiments, the low quality nucleic acid molecules have a DI of at least 1 and up to or less than 158.3.

In some of any of such embodiments, the nucleic acid sample comprises high quality nucleic acid molecules. In some of any of such embodiments, the high quality nucleic acid molecules have a DI of less than 1.

In some of any of such embodiments, the nucleic acid sample is a forensic sample. In some of any of such embodiments, the nucleic acid sample is derived from a buccal swab, paper, fabric, or other substrate that is impregnated with saliva, blood, or other bodily fluid.

In some of any of such embodiments, the nucleic acid sample comprises between or between about 50 μg and 100 ng of genomic DNA. In some of any of such embodiments, the nucleic acid sample comprises between or between about 100 pg and 5 ng of genomic DNA or between or between about 50 pg and 5 ng of genomic DNA. In some of any of such embodiments, the nucleic acid sample comprises at or about 1 ng of genomic DNA.

In some of any of such embodiments, the plurality of SNPs comprises kinship SNPs. In some of any of such embodiments, the plurality of SNPs comprises kiSNPs, biogeographical ancestry SNPs (aiSNPs), identity SNPs (iiSNPs), phenotype SNPs (piSNPs), x-chromosome SNPs (xSNPs), and y-chromosome SNPs (ySNPs). In some of any of such embodiments, the plurality of SNPs comprises SNPs selected from one or more of the groups consisting of kiSNPs, aiSNPs, iiSNPs, piSNPs, xSNPs, and ySNPs. In some of any of such embodiments, at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

Also provided herein is a method of identifying genetic relatives of a DNA profile, comprising: calculating the degree of relationship of the DNA profile generated using any of the methods provided herein to the one or more reference DNA profiles; and generating a family tree comprising the DNA profile in relation to the one or more reference DNA profiles.

In some of any of such embodiments, the method further comprises generating a family tree comprising the DNA profile in relation to one or more DNA profiles. In some of any of such embodiments, the one or more reference DNA profiles are part of a genealogy database.

In some of any of such embodiments, the one or more reference DNA profiles comprises at or about or at least or at least about 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles. In some of any of such embodiments, the one or more reference DNA profiles comprises at or about or at least or at least about 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles.

Also provided herein is a method of identifying genetic relatives of a DNA profile, comprising: calculating the degree of relationship of a DNA profile comprising genotypes of at least between at or about 5,000 to 50,000 SNPs to the one or more reference DNA profiles; and generating a family tree comprising the DNA profile in relation to the one or more reference DNA profiles. In some embodiments, the DNA is generated by the method of any one of claims 52-71.

Also provided herein is a kit comprising at least one container means, wherein the at least one container means comprises any of the plurality of primers as described herein. In some of any of such embodiments, the plurality of SNPs comprises between about 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs. In some of any of such embodiments, the plurality of SNPs comprises 10,230 SNPs. In some of any of such embodiments, the plurality of SNPs comprises between about 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs. In some of any of such embodiments, the plurality of SNPs comprises 10,230 SNPs.

In some of any of such embodiments, the method further comprises generating a family tree comprising the DNA profile in relation to one or more DNA profiles.

### BRIEF DESCRIPTION OF THE DRAWINGS

FIG. 1 depicts an exemplary schematic of the method of generating a library capable of being sequenced.

FIG. 2 shows the results of the number of loci identified using varying input titrations of genomic DNA including 5 ng, 2.5 ng, 1 ng, 500 pg, 250 μg, 100 pg and 50 pg.

FIG. 3 shows the percentage of Loci detected (call rate) with degraded DNA using the assay described herein compared to Microarray (GSA) call rate.

FIG. 4 shows the number of loci detected in the presence of inhibitors hematin, humic acid, indigo, and tannic acid, compared to a reference control.

FIG. 5 depicts an exemplary family tree generated by the methods described herein.

FIG. 6 shows the expected and observed Kinship Coefficients calculated using the algorithm described herein.

FIG. 7 shows the results of the 1:many search algorithm in an exemplary case study.

FIG. 8. depicts an exemplary family tree generated from the results of the 1:many search algorithm.

FIG. 9 is a table summarizing the number and type of loci detected using varying input titrations of genomic DNA, including 5 ng, 2.5 ng, 1 ng, 500 pg, 250 μg, 100 pg, and 50 pg.

FIG. 10 is a table summarizing the number and type of loci detected using DNA in the presence of the inhibitors hematin, humic acid, tannic acid, and indigo, compared to a positive amplification control, in the absence of inhibitors.

FIG. 11 is a table summarizing the number and type of loci detected for two samples of DNA obtained 9 hours and 22 hours after a mock sexual assault. The DNA was isolated from the sperm fraction of a differential extraction method, and had an input of 500 pg of DNA.

FIG. 12 shows the number of loci detected in saliva samples with an increasing content of phenol (a known PCR amplification inhibitor) from a phenol-chloroform-isoamyl alcohol (PCIA) extraction method.

FIG. 13 shows the number of loci detected in blood samples isolated from different substrates or methods typically performed in forensics laboratories, including blood with rust, blood in denim, blood on a swab, and blood with varying levels of heme (a known PCR amplification inhibitor) carry-over from Chelex™ extraction.

### DETAILED DESCRIPTION

The practice of the techniques described herein may employ, unless otherwise indicated, conventional techniques and descriptions of molecular biology, cell biology, biochemistry and sequencing technology, which are within the skill of those who practice in the art. Specific illustrations of suitable techniques can be had by reference to the examples herein.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Overview

Provided herein are new and improved methods for the generation of DNA based profile analysis, including the generation of nucleic acid profiles and DNA-based kinship analysis. Current methods of generating DNA profiles for comparisons in genetic databases include genotyping using dense SNP microarrays and WGS followed by association of evidentiary samples with distant relatives in databases, which require high quantity and high quality DNA samples, and are not designed for familial searching or forensic purposes. Forensic casework samples, e.g., from a crime scene, are generally low quantity and low quality samples, e.g., includes degraded DNA, and data from the current methods requires extensive imputation to generate results capable of being uploaded to a search database. Moreover, forensic samples often include agents that are inhibitors of PCR amplification reactions. The new and improved methods provided herein overcome these limitations by allowing for the use of low quantity and low quality, e.g., degraded, DNA for the generation of nucleic acid profiles, even when the samples include known inhibitors of PCR amplification, using primers that specifically hybridize to about 5,000 to 50,000 SNPs for a more efficient genetic analysis than alternative approaches like WGS or SNP microarrays. Moreover, the new and improved methods provided herein also include an improved method of performing kinship analysis that requires fewer computations for calculating accurate kinship. Finally, in some embodiments, the new and improved methods provided herein also exclude SNPs with known medical associations or low minor allele frequencies, which limits privacy concerns and protects genetic health data.

Disclosed herein are methods of performing DNA-based kinship analysis, which include providing a nucleic acid sample, and subsequently amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions. In some embodiments, a nucleic acid library (e.g., a DNA library) is generated from the amplification products. In some embodiments, the nucleic acid library generated from the amplification products is sequenced, and the genotypes of the plurality of SNPs are determined. In some embodiments, the amplification products are sequenced and amplified, and the genotypes of the plurality of SNPs are determined. In some embodiments, the genotypes of the plurality of SNPs are used to generate a DNA profile. In some embodiments, the degree of relationship of the DNA profile to one or more reference DNA profiles is determined.

In some embodiments, the methods disclosed herein comprise performing DNA-based kinship analysis, which includes providing a nucleic acid sample, and subsequently amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions. In some embodiments, a nucleic acid library, e.g., a DNA library, is generated from the amplification products. In some embodiments, the nucleic acid library, e.g., DNA library, generated from the amplification products is sequenced, and the genotypes of the plurality of SNPs are determined. In some embodiments, the amplification products are sequenced, and the genotypes of the plurality of SNPs are determined. In some embodiments, the genotypes of the plurality of SNPs are used to generate a DNA profile. In some embodiments, the degree of relationship of the DNA profile to one or more reference DNA profiles is determined.

In some embodiments, disclosed herein is a plurality of primers that specifically hybridize to a plurality of target sequences collectively comprising at least between at or about 5,000 to 50,000 single nucleotide polymorphisms (SNPs) in a nucleic acid sample, wherein amplifying the nucleic acid sample using the plurality of primers in one or more multiplex reactions results in amplification products.

In some embodiments, the methods disclosed herein comprise constructing a nucleic acid library, which includes providing a nucleic acid sample, and subsequently amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions. In some embodiments, the amplification products are sequenced, and the genotypes of the plurality of SNPs are determined. In some embodiments, the genotypes of the plurality of SNPs are used to generate a DNA profile.

In some embodiments, the methods disclosed herein comprise constructing a DNA profile, which includes providing a nucleic acid sample, and subsequently amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions. In some embodiments, the amplification products are sequenced, and the genotypes of the plurality of SNPs are determined. In some embodiments, the genotypes of the plurality of SNPs are used to generate a DNA profile.

In some embodiments, the methods described herein comprise identifying genetic relatives of a DNA profile, which includes calculating the degree of relationship of a DNA profile comprising genotypes of at least between at or about 5,000 to 50,000 SNPs to the one or more reference DNA profiles; and generating a family tree comprising the DNA profile in relation to the one or more reference DNA profiles.

### Samples and Sample Processing

In some aspects, the sample disclosed herein can be or comprise any suitable biological sample, or a sample derived therefrom. In some aspects, the samples described herein are processed and amplified using any known suitable method to complement the methods described herein. Exemplary samples, methods of sample processing and methods of sample amplification are described below.

A. Nucleic Acid Samples

A nucleic acid sample disclosed herein can be derived from any biological sample. A biological sample may be derived from blood, buccal swabs, hair, teeth, bone, and/or semen. In some embodiments, the nucleic acid sample is derived from a biological sample that is or comprises blood, hair, teeth, bone, semen, or sperm. In some embodiments, the biological sample is a DNA sample. In some embodiments, the nucleic acid sample comprises DNA. In some embodiments, the DNA is genomic DNA (gDNA). The DNA from which the nucleic acid sample may be obtained may be intact or partially degraded. The DNA from which the nucleic acid sample may be obtained may be compromised, degraded or inhibited due, but not limited to, to source material age, variable extraction, storage procedures or environmental exposure. In some embodiments, the DNA is compromised due to calcium inhibition, cremation, burning, and embalming.

In some embodiments, the DNA from which the nucleic acid sample is obtained is a low quantity and/or low quality DNA sample. In some embodiments, the DNA from which the nucleic acid sample is obtained is a low quantity and low quality DNA sample. In some embodiments, the low quality DNA sample comprises low quality nucleic acid molecules. In some embodiments, the low quality nucleic acid molecules are degraded DNA, e.g., genomic DNA, and/or are fragmented DNA, e.g., genomic DNA.

The quality of a nucleic acid, e.g., DNA, sample can be determined by calculating a degradation index (DI). DI is calculated by dividing the concentration of small DNA targets by the concentration of large DNA targets (DI=concentration of small DNA targets/concentration of large DNA targets). In general, a DI value of less than 1 typically indicates that the nucleic acid, e.g., DNA, is not degraded, is not a low quality sample, and/or is a high quality sample; a DI value of 1 to 10 typically indicates that the nucleic acid, e.g., DNA, has a minor to moderate amount of degradation; and a DI value of greater than 10 typically indicates that the nucleic acid, e.g., DNA, is highly degraded.

In some embodiments, the low quality nucleic acid molecules have a DI of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 or more. In some embodiments, the low quality nucleic acid molecules have a DI of at least 1 and at or less than 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 or more. In some embodiments, the low quality nucleic acid molecules have a DI of between 1 and 200. In some embodiments, the low quality nucleic acid molecules have a DI of at least 1 and at or less than 158.3.

In some embodiments, the DNA from which the nucleic acid sample is obtained is a high quality nucleic acid sample. In some embodiments, the high quality nucleic acid sample has a DI of less than 1.

In some embodiments, the nucleic acid sample comprises one or more enzyme inhibitors. In some embodiments, the one or more enzyme inhibitors comprise one or more inhibitors selected from the group consisting of hematin, humic acid, indigo, and tannic acid. In some embodiments, the one or more enzyme inhibitors comprises heme.

In some embodiments, the nucleic acid sample is a forensic sample. In some embodiments, the nucleic acid sample is derived from a buccal swab, paper, fabric, e.g., denim, or other substrate that is impregnated with saliva, blood, sperm, or other bodily fluid.

In some embodiments, the nucleic acid sample is from a crime scene, such as a homicide, an assault, such as a sexual assault, or a burglary, or any other crime where identification of a participant is needed. In some embodiments, the nucleic acid sample is from a sexual assault.

In some embodiments, the nucleic acid sample is obtained at or about 30 minutes, at or about 1 hour, or at or about 2, 3, 4, 5, 6, 7, 8, 9, 10, 11, 12, 13, 14, 15, 16, 17, 18, 19, 20, 21, 22, 23, or 24 or more hours after a sample containing the nucleic acid sample was deposited by its source, e.g., a human subject. In some embodiments, the nucleic acid sample is obtained at or less than about 3 hours, 9 hours, 12 hours, 15 hours, 18 hours, 21 hours, 22 hours, 24 hours, 36 hours, 48 hours, 3 days, 4 days, 5 days, 6 days, 7 days, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, or 4 or more years after a sample containing the nucleic acid sample was deposited by its source, e.g., a human subject. In some embodiments, the nucleic acid sample is obtained at or less than 24 hours, e.g., at or less than 22 hours, after a sample containing the nucleic acid sample was deposited by its source, e.g., a human subject.

In some embodiments, the nucleic acid sample comprises between or between about 50 pg and 100 ng of DNA, e.g., genomic DNA. In some embodiments, the nucleic acid sample comprises between or between about 100 pg and 5 ng of DNA, e.g., genomic DNA. In some embodiments, the nucleic acid sample comprises at or about 1 ng of DNA, e.g., genomic DNA.

In some embodiments, the nucleic acid sample comprises at or about 10 pg to at or about 100 ng of DNA, e.g., genomic DNA, or comprises at or about 10 pg to at or about 5 ng of DNA, e.g., genomic DNA. In some embodiments, the nucleic acid sample comprises at or about 10 pg to 10 ng, at or about 10 pg to 5 ng, at or about 25 pg to 10 ng, at or about 25 pg to 5 ng, at or about 50 pg to 10 ng, or at or about 50 pg to 5 ng, of DNA, e.g., genomic DNA.

In some embodiments, the nucleic acid sample comprises at or about 50 pg to at or about 5 ng of DNA, e.g., genomic DNA. In some embodiments, the nucleic acid sample comprises at or about 10 pg, 15 pg, 20 pg, 25 pg, 30 pg, 35 pg, 40 pg, 45 pg, 50 μg, 55 pg, 60 pg, 70 pg, 75 pg, 80 pg, 85 pg, 90 pg, 95 pg, 100 pg, 125 pg, 150 μg, 175 pg, 200 pg, 225 pg, 250 μg, 275 pg, 300 pg, 325 pg, 350 μg, 375 pg, 400 pg, 420 pg, 425 pg, 450 μg, 475 pg, 500 pg, 600 pg, 700 pg, 800 pg, 900 pg, 1 ng, 1.1 ng, 1.2 ng, 1.3 ng, 1.4 ng, 1.5 ng, 1.6 ng, 1.7 ng, 1.8 ng, 1.9 ng, 2 ng, 2.1 ng, 2.2 ng, 2.3 ng, 2.4 ng, 2.5 ng, 2.6 ng, 2.7 ng, 2.8 ng, 2.9 ng, 3 ng, 3.25 ng, 3.5 ng, 3.75 ng, 4 ng, 4.25 ng, 4.5 ng, 4.75 ng, or 5 ng of DNA, e.g., genomic DNA, or between any two preceding values. In some embodiments, the nucleic acid sample comprises between or between about 10 pg and 10 ng, between or between about 10 pg and 5 ng, between or between about 10 pg and 4 ng, between or between about 10 pg and 3 ng, between or between about 10 pg and 2 ng, between or between about 25 pg and 10 ng, between or between about 25 pg and 5 ng, between or between about 25 pg and 4 ng, between or between about 25 pg and 3 ng, between or between about 25 pg and 2 ng, between or between about 40 pg and 10 ng, between or between about 40 pg and 5 ng, between or between about 40 pg and 4 ng, between or between about 40 pg and 3 ng, between or between about 40 pg and 2 ng, between or between about 50 μg and 10 ng, between or between about 50 μg and 5 ng, between or between about 50 μg and 4 ng, between or between about 50 μg and 3 ng, between or between about 50 μg and 2 ng, between or between about 10 pg and 2 ng, between or between about 10 pg and 1.5 ng, between or between about 10 pg and 1 ng, between or between about 20 pg and 2 ng, between or between about 20 pg and 1.5 ng, between or between about 20 pg and 1 ng, 25 pg and 2 ng, between or between about 25 pg and 1.5 ng, between or between about 25 pg and 1 ng, between or between about 30 pg and 2 ng, between or between about 30 pg and 1.5 ng, between or between about 30 pg and 1 ng, between or between about 35 pg and 2 ng, between or between about 35 pg and 1.5 ng, between or between about 35 pg and 1 ng, between or between about 40 pg and 2 ng, between or between about 40 pg and 1.5 ng, between or between about 40 pg and 1 ng, between or between about 45 pg and 2 ng, between or between about 45 pg and 1.5 ng, between or between about 45 pg and 1 ng, between or between about 50 μg and 2 ng, between or between about 50 pg and 1.5 ng, or between or between about 50 μg and 1 ng.

B. Sample Processing and Amplification

A variety of steps can be performed to prepare or process a nucleic acid sample for and/or during an assay. Except where indicated otherwise, the preparative or processing steps described below can generally be combined in any manner and in any order to appropriately prepare or process a particular sample for analysis and/or sequencing, disclosed herein.

In some embodiments, the amount of the nucleic acid sample provided is, is about, or is less than 1 ng of genomic DNA. In some embodiments, the methods disclosed herein comprise amplification of the genomic DNA. In some embodiments, amplification of the genomic DNA includes one or more multiplex polymerase chain reactions (PCR) comprising a plurality of primers, thereby generating amplification products. In some embodiments, amplification of the genomic DNA includes a single multiplex PCR reaction. In some embodiments, amplification of the genomic DNA includes two multiplex PCR reactions. In some embodiments, amplification of the genomic DNA includes three multiplex PCR reactions. In some embodiments, amplification of the genomic DNA includes four multiplex PCR reactions.

In some embodiments, one or more primers in the plurality of primers are designed in accordance with the atypical design strategy as described in WO 2015/126766 A1, which is hereby incorporated by reference in its entirety. In some embodiments, one or more primers in the plurality of primers is at least 24 nucleotides in length, and/or has a melting temperature that is less than 60 degrees C., and/or is AT-rich with an AT content of at least 60%. In some embodiments, one or more primers in the plurality of primers comprises a length of at least 24 nucleotides that hybridize to the target sequence, and/or has a melting temperature that is between 50 degrees C. and 60 degrees C., and/or is AT-rich with an AT content of at least 60%. In some embodiments, one or more primers in the plurality of primers has a melting temperature that is less than 58 degrees C., or is less than 54 degrees C.

In some embodiments, the genomic DNA may be amplified for a number of cycles using the plurality of primers that hybridize and/or tag a plurality of target sequences collectively comprising a plurality of at least between at or about 5,000 to 50,000 single nucleotide polymorphisms (SNPs). In some embodiments, the genomic DNA may be amplified for a number of cycles using the plurality of primers that hybridize and/or tag a plurality of target sequences collectively comprising at least between at or about 5,000 to 15,000, 20,000, 25,000, 30,000, 35,000, 40,000, 45,000, or 50,000 SNPs. In some embodiments, the genomic DNA may be amplified for a number of cycles using the plurality of primers that hybridize and/or tag a plurality of target sequences collectively comprising at least between at or about 10,000 to 11,000 SNPs. In some embodiments, the genomic DNA may be amplified for a number of cycles using the plurality of primers that hybridize and/or tag a plurality of target sequences collectively comprising at least between at or about 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs. In some embodiments, the genomic DNA may be amplified for a number of cycles using the plurality of primers that hybridize and/or tag a plurality of target sequences collectively comprising at or about 10,230 SNPs.

In some embodiments, the plurality of SNPs comprises at least between at or about 5,000 to 15,000, 20,000, 25,000, 30,000, 35,000, 40,000, 45,000, or 50,000 SNPs. In some embodiments, the plurality of SNPs comprises at least between at or about 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs. In some embodiments, the plurality of SNPs comprises at or about 10,230 SNPs. In some embodiments, the plurality of SNPs comprises at least between at or about 5,000 to 50,000 SNPs, 5,000 to 45,000 SNPs, 5,000 to 40,000 SNPs, 5,000 to 35,000 SNPs, 5,000 to 30,000 SNPs, 5,000 to 25,000 SNPs, 5,000 to 20,000 SNPs, 6,000 to 50,000 SNPs, 6,000 to 45,000 SNPs, 6,000 to 40,000 SNPs, 6,000 to 35,000 SNPs, 6,000 to 30,000 SNPs, 6,000 to 25,000 SNPs, 6,000 to 20,000 SNPs, 7,000 to 50,000 SNPs, 7,000 to 45,000 SNPs, 7,000 to 40,000 SNPs, 7,000 to 35,000 SNPs, 7,000 to 30,000 SNPs, 7,000 to 25,000 SNPs, 7,000 to 20,000 SNPs, 8,000 to 50,000 SNPs, 8,000 to 45,000 SNPs, 8,000 to 40,000 SNPs, 8,000 to 35,000 SNPs, 8,000 to 30,000 SNPs, 8,000 to 25,000 SNPs, 8,000 to 20,000 SNPs, 9,000 to 50,000 SNPs, 9,000 to 45,000 SNPs, 9,000 to 40,000 SNPs, 9,000 to 35,000 SNPs, 9,000 to 30,000 SNPs, 9,000 to 25,000 SNPs, or 9,000 to 20,000 SNPs.

In some embodiments, the plurality of SNPs comprises SNPs selected from one or more of the groups consisting of kinship SNPs, ancestry SNPs, identity SNPs, phenotype SNPs, X-SNPs, and Y-SNPs. In some embodiments, the plurality of SNPs comprises kinship SNPs, ancestry SNPs, identity SNPs, phenotype SNPs, X-SNPs, and Y-SNPs. In some embodiments, the plurality of SNPs comprises kinship SNPs.

In some embodiments, the SNPs do not include SNPs with known medical associations, e.g., associated with known medical conditions, or low minor allele frequencies. By excluding SNPs with known medical associations, e.g., associated with known medical conditions, or low minor allele frequencies, privacy concerns are limited and genetic health data is protected.

In some embodiments, the SNPs comprise SNPs that have been filtered with a plurality of genotype samples. In some embodiments, the SNPs are selected from categories including ancestry SNPs, identity SNPs, kinship SNPs, phenotype SNPs, X-SNPs and Y-SNPs. In some embodiments, the ancestry SNPs include between at or about 10-100 SNPs. In some embodiments, the identity SNPs include between at or about 10-200 SNPs. In some embodiments, the kinship SNPs include between at or about 7,000-12,000 SNPs. In some embodiments, the phenotype SNPs include between at or about 1-50 SNPs. In some embodiments, the X-SNPs include between at or about 10-200 SNPs. In some embodiments, the Y-SNPs include between at or about 10-200 SNPs. In some embodiments, the ancestry SNPs include between at or about 0-10% of the total number of SNPs. In some embodiments, the identity SNPs include between at or about 0-10% of the total number of SNPs. In some embodiments, the kinship SNPs include between at or about 80-100% of the total number of SNPs. In some embodiments, at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs. In some embodiments, 100% of the plurality of SNPs are kinship SNPs. In some embodiments, the phenotype SNPs include between at or about 0-5% of the total number of SNPs. In some embodiments, the X-SNPs include between at or about 0-5% of the total number of SNPs. In some embodiments, the Y-SNPs include between at or about 0-5% of the total number of SNPs. In some embodiments, the SNPs do not include medically informative or minor allele frequency SNPs. A tag region can be any sequence, such as a universal tag region, a capture tag region, an amplification tag region, a sequencing tag region, a UNIT tag region, and the like.

In some embodiments, target sequences are purified and enriched, and a library of the original DNA sample, also referred to as a nucleic acid library, is generated. In some embodiments, the purification combines purification beads with an enzyme to purify the amplified targets from other reaction components. In some embodiments, the purified target sequences are enriched by amplification of the DNA and addition of UDI adapters and sequences required for cluster generation. The UDI adapters can tag DNA with a unique combination of sequences that identify each sample for analysis.

In some embodiments, a nucleic acid library is generated from the amplification products, including the amplification products produced by any of the methods or embodiments described herein. As such, in some embodiments, the nucleic acid library comprises the amplification products generated by amplifying the nucleic acid sample with the plurality of primers that specifically hybridize to a plurality of target sequences collectively comprising a plurality of at least between at or about 5,000 to 50,000 SNPs.

In some embodiments, nucleic acid libraries or DNA libraries are normalized to quantify and check for quality, and pooled by combining equal volumes of normalized libraries to create a pool of libraries capable of being sequenced together on the same flow cell. In some embodiments, the quantification includes the use of a fluorimetric method. In some embodiments, the quantification includes a quantitative PCR method. After the DNA libraries are pooled, they can be denatured and diluted using a sodium hydroxide (NaOH)-based method, and a sequencing control can be added.

In some embodiments, the nucleic acid libraries are quantitated, normalized, denatured and diluted as per instructions given in Forenseq Kintelligence kit User Guide (Verogen PN:V16000120, the contents of which are hereby incorporated by reference in their entirety).

In some embodiments, the nucleic acid libraries of DNA libraries are prepared for sequencing using massively parallel sequencing using any known suitable method to complement the methods described herein.

### Sequencing and Analysis

In some aspects, the nucleic acid libraries or DNA libraries described in Section II herein can be sequenced using any known suitable method to complement the methods described herein, and are not limited to any particular sequencing platform. In some aspects, the sample disclosed herein can be analyzed using any known suitable method to complement the methods described herein. Exemplary methods of sequencing and methods analysis are described below.

A. Sequencing

In some embodiments, the technology for sequencing the nucleic acid libraries or DNA libraries created by practicing the methods described herein comprise the use of polymerase-based sequencing by synthesis, ligation based, pyrosequencing or polymerase-based sequencing methods.

In some embodiments, the nucleic acid library is sequenced as per instructions on MiSeq FGx Sequencing System Reference Guide (document #VD2018006, the contents of which are hereby incorporated by reference in their entirety). In some embodiments, the nucleic acid library that is sequenced as per instructions on MiSeq FGx Sequencing System Reference Guide (document #VD2018006) is denatured.

In some aspects, the sequencing methods disclosed herein comprise the use of massively parallel sequencing (MPS). In some aspects, the sequencing methods disclosed herein do not comprise the use of whole genome sequencing (WGS). In some aspects, the sequencing methods disclosed herein do not comprise the use of microarrays.

In some embodiments, the sequencing methods disclosed herein detect at or about 90% of the loci of the SNPs.

In some embodiments, the sequencing methods disclosed herein generate an output report comprising the results of the sequencing of the amplification products comprising the plurality of SNPs.

B. Analysis

In some aspects, the methods disclosed herein involve the use of an analysis module that automatically initiates analysis once the sequencing of the samples (i.e. amplification products) is complete. In some embodiments, the analysis module includes Universal Analysis Software (UAS).

In some embodiments, the analysis methods disclosed herein generate an output report comprising the results of the sequencing of the amplification products comprising the plurality of SNPs.

In some embodiments, sequencing results are analyzed using any suitable sequence analysis software available in the art.

In some embodiments, sequencing results are analyzed using the Forenseq Universal Analysis Software, such as version 2.1 or 2.2 or later (Verogen, San Diego, CA) following the instructions outlined in a Forenseq Universal Analysis Software Reference Guide, such as for version 2.2 or later, and provided in, e.g., Reference Guide Document #VD2019002, the contents of which are hereby incorporated by reference in their entirety.

### Genotype and DNA Profile Determination

In some aspects, the output report comprising the results of the sequencing of the amplification products comprising the plurality of SNPs generated by any of the methods described herein can be used to genotype the sample using any known suitable method to complement the methods described herein. In some aspects, the output report comprising the results of the sequencing of the amplification products comprising the plurality of SNPs generated by any of the methods described herein can be used to generate a DNA profile using any known suitable method to complement the methods described herein.

In some embodiments, the DNA profile includes a genotype for each of the plurality of SNPs. In some embodiments, the DNA profile includes a genotype for at least or at least about 80%, 85%, 90%, 91%, 92%, 93%, 94%, 95%, 96%, 97%, 98%, or 99% of the plurality of SNPs. In some embodiments, the DNA profile includes a genotype for at least or at least about 99% or about 100% of the SNPs.

In some embodiments, the methods disclosed herein include determination of hair color, eye color and biogeographical ancestry.

### Degree of Relationship Determination

In some aspects, the degree of relationship of the DNA profile described in Section IV herein can be calculated with reference to one or more reference DNA profiles using any known suitable method to complement the methods described herein.

In some embodiments, the DNA-based kinship analysis described herein includes the use of GEDmatch PRO. In some embodiments, the DNA-based kinship analysis described herein allows for generation of a report with minimal user input. In some embodiments, the DNA-based kinship analysis described herein comprises the use of an algorithm to calculate kinship coefficient. In some embodiments, the kinship coefficient determines the relationship status of the sample or DNA profile to a reference DNA profile on a database. For instance, in some embodiments, the kinship coefficient indicates whether each of the one or more identified genetic relatives is likely to be a great great grandmother, a great great grandfather, a great grandfather, a great grandmother, a grandmother, a grandfather, a first cousin, a first cousin once removed, or a second cousin, based on the relative value of the kinship coefficient. In some embodiments, the reference DNA profiles are part of a genealogy database.

In some embodiments, the DNA-based kinship analysis described herein comprises identifying genetic relatives to at or about the 1^{st}, 2^{nd} 3^{rd} 4^{th} or 5^{th} degree. In some embodiments, the DNA-based kinship analysis described herein comprises identifying genetic relatives to more than the 1^{st} 2^{nd} 3^{rd} 4^{th} or 5^{th} degree.

In some embodiments, the DNA-based kinship analysis described herein comprises generating a family tree comprising the DNA profile in relation to one or more DNA profiles. The family tree can be generated using any available means or methodologies.

In some embodiments, the DNA-based kinship analysis described herein comprises identifying suspects through common ancestors.

In some embodiments, the calculating the degree of relationship comprises the use of a principal component analysis (PCA) method. In some embodiments, the calculating the degree of relationship comprises the use of the PC-Relate method. See, e.g., Conomos et al., Model-free Estimation of Recent Genetic Relatedness, Am. J. Hum. Genet., 98(1): 127-148 (2016). In some embodiments, the calculating the degree of relationship comprises the use of principal component analysis in related samples (PC-AiR).

The PC-AiR method allows for ancestry determination in the presence of known or cryptic relatedness. See, e.g., Conomos et al., Robust Inference of Population Structure for Ancestry Prediction and Correction of Stratification in the Presence of Relatedness, Genet Epidemiol., 2015, 39(4): 276-293, the contents of which are hereby incorporated by reference. However, PC-Relate and PC-AiR were developed during an era when researchers were routinely dealing with determining relatedness using hundreds to the low thousands of samples (e.g., reference DNA profiles), rather than having access to a database of, e.g., tens of thousands of samples, hundreds of thousands of samples, or even more than 1 million samples, e.g., ˜1.5 million samples, as researchers today have access to. Thus, when building models for calculating the degree of relatedness, of which picking unrelated individuals is a part, there has been both a drive to include as many samples as possible, and far less pressure to be computationally efficient.

The PC-AiR method is feasible for calculating the degree of relatedness when the total number of samples (e.g., reference DNA profiles) is small, e.g., less than 5,000 samples, but when it is scaled to a significantly larger number of samples, such as with forensic databases or kinship databases, it requires either: (a) massive amounts of computation that scale exponentially with the number of samples, or (b) use of a different data structure to reduce computational complexity at the cost of requiring massive amounts of memory, e.g., random access memory (RAM). Moreover, PC-AiR's development was likely driven by the need to do its analysis in GWAS studies where users would generally need to perform the analysis only once or a handful of times after data collection, e.g., milestones, rather than on an on-going basis as is required by an ever-growing database for forensics. Accordingly, the PC-AiR method is not feasible for use with more than a small number of samples, e.g., less than 5,000 samples, such as with tens of thousands of samples, hundreds of thousands of samples, or even 1 million or more samples, e.g., ˜1.5 million samples, due to, among other things, the substantial increase in computational resources and time required to complete the analysis. Therefore, an alternative approach to the PC-AiR method is described herein for use with a large number of samples, e.g., at least 5,000 samples, that allows for lower computational complexity than PC-AiR while providing an acceptable result on the relatedness of the DNA profile to reference DNA profiles. This alternative approach is referred to herein as the “large cohort” method. The large cohort method is intended to be used when the sample number, e.g., the number of reference DNA profiles, is very large and, thus, there is less of a need to include as many samples as possible when building the model due to the large number of samples available, thereby allowing for computational efficiency to be achieved.

In some embodiments, the PC-AiR method and the large cohort method described herein both include an unrelated set picking process that starts and ends at the same places, with: (1) an input that is a cohort of samples with genotypes at a chosen set of SNPs, e.g., the plurality of SNPs; and (2) an output that is the identities of a set of mutually unrelated samples within the input cohort of samples. The goal of the process is to identify a sufficiently acceptable unrelated sample set that is as close to as large as possible while also sampling well from all ancestral backgrounds present in the cohort of samples.

In some embodiments, the PC-AiR method and the large cohort method described herein both include the same initial step of using a simplified kinship estimation method called “KING-Robust” to estimate kinship between all pairs of samples. This step is computationally complex. The PC-AiR method and the large cohort method then diverge, and the PC-AiR method then proceeds into subsequent steps that are very high complexity: (1) initializing a set “U” with all of the samples; (2) scanning the set to calculate, for each sample, how many samples that sample is related to in U (referred to as “R”), and how many samples it is “ancestrally diverged” from in U (referred to as “D”); (3) selecting the sample with the highest U and, if there are multiple samples having the highest U, then selecting the sample having the highest U and the lowest D and then removing it from U; and (4) repeating from step 2. For instance, using the PC-AiR method, if there are 50,000 samples, the process will look at 50,000² data points in the first iteration, 49,999² data points in the second iteration, and so on until there are no more related samples in the set, which may proceed down to, e.g., 20,000² or 10,000² data points. Thus, the PC-AiR method may be feasible when the total number of samples is small, e.g., 2,000, but when it is scaled to a significantly larger number of samples, e.g., 10,000, or 50,000, or 100,000 or more samples, it requires either: (a) massive amounts of computation that scale exponentially with the number of samples, or (b) use of a different data structure to reduce computational complexity at the cost of requiring massive amounts of memory, e.g., RANI. As such, the PC-AiR method is not feasible when using a large number of samples, e.g., 10,000 or more, with a desire to have results within a matter of minutes to hours (rather than days to months) due to the computational complexity, resources, and extended amount of time required with the PC-AiR method.

Accordingly, in some embodiments, the calculating the degree of relationship comprises the use of a large cohort method (which is an alternative approach to PC-AiR suitable for large sample sizes) that comprises the following adjustments to the PC-AiR method: (1) redefining “related” to be more stringent by, e.g., specifically using a KING-Robust kinship>0.01 instead of >0.025 as in the PC-AiR method; (2) remove all samples with >5% missing genotypes (e.g., more than 5% of the SNPs in the reference DNA profile) in order to make sure that each sample is sufficiently informative; (3) for each sample, compute: “R” which is the total number of related samples in the total data set, “D” which is the number of ancestral diverged samples in the dataset, and “S” which is the set of related samples; (4) rank all samples by R (ascending) and D (descending); (5) iterate through the ranked list of samples and: (i) if the sample is not yet in the “related” set, add it to the unrelated set and add all samples from S (i.e., reference DNA profiles related to the DNA profile) to the related set; or (ii) if the sample is in the “related” set, disregard the sample and move to the next sample. This large cohort method allows for a process that is largely linear complexity (i.e., the runtime expands linearly with the number of samples) rather than exponential, and is, therefore, tractable on much larger sample cohorts than what PC-AiR could be used with, e.g., with at least 5,000 or more reference DNA profiles.

In some embodiments, the calculating the degree of relationship comprises the use of a modified form of PC-AiR comprising: (1) redefining “related” to be more stringent by, e.g., specifically using a KING-Robust kinship>0.01 instead of >0.025 as in the PC-AiR method; and (2) remove all samples with >5% missing genotypes (e.g., more than 5% of the SNPs in the reference DNA profile). In some embodiments, the modified form of PC-AiR further comprises, (a) for each sample, computing: “R” which is the total number of related samples in the total data set, “D” which is the number of ancestral diverged samples in the dataset, and “S” which is the set of related samples; (b) ranking all samples by R (ascending) and D (descending); and (c) iterating through the ranked list of samples and, in some embodiments: (i) if the sample is not yet in the “related” set, adding it to the unrelated set and add all samples from S (i.e., reference DNA profiles related to the DNA profile) to the related set; or (ii) if the sample is in the “related” set, disregard the sample and move to the next sample.

In some embodiments, the calculating the degree of relationship comprises a PC-AiR method. In some embodiments, the PC-AiR method comprises the steps of: (1) performing a KING-Robust kinship estimation between all pairs of a sample set comprising samples comprising the one or more reference DNA profiles, wherein parings with a kinship coefficient>0.025 are identified as related and parings with a kinship coefficient<−0.025 are identified as ancestry-diverged; (2) initializing an unrelated sample set as including all samples; and (3) iteratively: (i) identifying the set in the unrelated sample set that have the most related samples in the unrelated sample set, thereby designated as X, (ii) identifying the set of samples in X that have at least ancestry-diverged pairings compared to samples in the unrelated sample set, thereby designated as Y; and (iii) if Y has zero samples, then terminate the process, or, if Y has at least one sample, then randomly select one sample from Y to remove from U, and repeat beginning at step (3)(i).

In some embodiments, the calculating the degree of relationship comprises a large cohort method comprising the steps of: (1) performing a KING-Robust kinship estimation between all pairs of a sample set comprising the one or more reference DNA profiles, wherein parings with a kinship coefficient>0.01 are identified as related and parings with a kinship coefficient<−0.025 are identified as ancestry-diverged; (2) removing all reference DNA profiles that have≥5% missing data; (3) rank all reference DNA profiles by identifying each reference DNA profile with a ranking value. In some embodiments, the ranking value is determined based on the number of related reference DNA profiles in the full set of reference DNA profiles that is ranked from least to most and ties are broken by the number of ancestry-diverged reference DNA profiles in the full set of reference DNA profiles as ranked from most to least; and iteratively through the ranked reference DNA profiles, for each reference DNA profile: (i) if the reference DNA profile is not yet in a related sample set, add it to an unrelated sample set and add all related reference DNA profiles to the related sample set, and (ii) if the reference DNA profile is already in the related sample set, then skip to the next reference DNA profile, and repeat beginning at step (3)(i).

In some embodiments, the one or more reference DNA profiles comprises between 1 and 10 million or more reference DNA profiles. In some embodiments, the one or more reference DNA profiles comprises at or about or at least or at least about 1, 5, 25, 50, 75, 100, 500, 1,000, 1,500, 2,000, 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles, or a range between any two of the preceding values. In some embodiments, the one or more reference DNA profiles comprises up to or up to about 100, 500, 1,000, 1,500, 2,000, 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles. In some embodiments, the one or more reference DNA profiles comprises between 5,000 and 500,000, or between 10,000 and 500,000, or between 15,000 and 500,000, or between 20,000 and 500,000, or between 25,000 and 500,000, or between 25,000 and 400,000, or between 25,000 and 300,000, or between 25,000 and 250,000, or between 50,000 and 500,000, or between 50,000 and 400,000, or between 50,000 and 300,000, or between 50,000 and 250,000 reference DNA profiles.

In some embodiments, the calculating the degree of relationship comprises the use of a PC-AiR method and the one or more reference DNA profiles comprises at least 1 and up to 100, 200, 300, 400, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 4,000, 4,500, or 5,000 reference DNA profiles, or a range between any two of the preceding values.

In some embodiments, the calculating the degree of relationship comprises the use of the large cohort method and the one or more reference DNA profiles comprises at or about or at least or at least about 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles, or a range between any two of the preceding values.

### Kits

Provided herein are kits comprising any of the primers, reagents or compositions described herein, which may further comprise instruction(s) on methods of using the kit, such as uses described herein. The kits described herein may also include other materials desirable from a commercial and user standpoint, including other buffers, diluents, filters, and package inserts with instructions for performing any methods described herein.

### Exemplary Embodiments

Among the exemplary embodiments provided herein are:
1. A method for performing DNA-based kinship analysis, comprising:
   providing a nucleic acid sample,
   amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions,
   generating a nucleic acid library from the amplification products,
   sequencing the nucleic acid library generated from the amplification products,
   analyzing the sequences of the amplification products,
   determining the genotypes of the plurality of SNPs, thereby generating a DNA profile, and
   calculating the degree of relationship of the DNA profile to one or more reference DNA profiles.
2. A method for performing DNA-based kinship analysis, comprising:
   providing a nucleic acid sample,
   amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions,
   generating a nucleic acid library from the amplification products
   sequencing the nucleic acid library generated from the amplification products,
   determining the genotypes of the plurality of SNPs, thereby generating a DNA profile, and
   calculating the degree of relationship of the DNA profile to one or more reference DNA profiles.
3. The method of embodiment 1 or embodiment 2, wherein the sequencing is conducted using massively parallel sequencing (MPS).
4. The method of any one of embodiments 1-3, wherein the sequencing does not comprise whole genome sequencing (WGS).
5. The method of any one of embodiments 1-4, further comprising generating a family tree comprising the DNA profile in relation to one or more DNA profiles.
6. A method of constructing a nucleic acid library, comprising:
   providing a nucleic acid sample,
   amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating a nucleic acid library comprising amplification products, wherein the amplification is carried out in one or more multiplex PCR reactions.
7. The method of any one of embodiments 1-6, wherein the nucleic acid sample comprises genomic DNA.
8. The method of any one of embodiments 1-7, wherein the nucleic acid sample comprises one or more enzyme inhibitors.
9. The method of embodiment 8, wherein the one or more enzyme inhibitors comprise one or more inhibitors selected from the group consisting of hematin, heme, humic acid, indigo, and tannic acid.
10. The method of any one of embodiments 1-9, wherein the nucleic acid sample comprises low-quality nucleic acid molecules and/or low quantity nucleic acid molecules.
11. The method of embodiment 10, wherein the low quality nucleic acid molecules are degraded genomic DNA and/or fragmented genomic DNA.
12. The method of embodiment 10 or embodiment 11, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.
13. The method of embodiment 10 or embodiment 11, wherein the low quality nucleic acid molecules have a DI of at least 1 and up to or less than 158.3.
14. The method of any one of embodiments 1-9, wherein the nucleic acid sample comprises high quality nucleic acid molecules.
15. The method of embodiment 14, wherein the high quality nucleic acid molecules have a DI of less than 1.
16. The method of any one of embodiment 1-15, wherein the nucleic acid sample is a forensic sample.
17. The method of any one of embodiments 1-16, wherein the nucleic acid sample is derived from saliva, blood, semen, hair, teeth, or bone.
18. The method of embodiment 17, wherein the nucleic acid sample is derived from saliva, blood, or semen.
19. The method of any one of embodiments 1-16, wherein the nucleic acid sample is derived from a buccal swab, paper, fabric, or other substrate that is impregnated with saliva, blood, semen, or other bodily fluid.
20. The method of any one of embodiments 1-19, wherein the nucleic acid sample comprises between or between about 50 μg and 100 ng of genomic DNA.
21. The method of any one of embodiments 1-20, wherein the nucleic acid sample comprises between or between about 100 pg and 5 ng of genomic DNA or between or between about 50 pg and 5 ng of genomic DNA.
22. The method of embodiment 20 or embodiment 21, wherein the nucleic acid sample comprises at or about 1 ng of genomic DNA.
23. The method of any one of embodiments 1-22, wherein the plurality of SNPs comprises kinship SNPs (kiSNPs).
24. The method of any one of embodiments 1-23, wherein the plurality of SNPs comprises kiSNPs, biogeographical ancestry SNPs (aiSNPs), identity SNPs (iiSNPs), phenotype SNPs (piSNPs), x-chromosome SNPs (xSNPs), and y-chromosome SNPs (ySNPs).
25. The method of any one of embodiments 1-23, wherein the plurality of SNPs comprises SNPs selected from one or more of the groups consisting of kiSNPs, aiSNPs, iiSNPs, piSNPs, xSNPs, and ySNPs.
26. The method of any one of embodiments 23-25, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.
27. A method for calculating degree of relatedness, comprising:
   obtaining a DNA profile comprising genotypes of at least between at or about 5,000 to 50,000 SNPs; and
   calculating the degree of relationship of the DNA profile to one or more reference DNA profiles.
28. A method for calculating degree of relatedness, comprising:
   generating a DNA profile comprising genotypes of at least between at or about 5,000 to 50,000 SNPs; and
   calculating the degree of relationship of the DNA profile to one or more reference DNA profiles.
29. The method of any one of embodiments 1-28, wherein the calculating the degree of relationship comprises a large cohort method comprising the steps of: (1) performing a KING-Robust kinship estimation between all pairs of a sample set comprising the one or more reference DNA profiles, wherein parings with a kinship coefficient>0.01 are identified as related and parings with a kinship coefficient<−0.025 are identified as ancestry-diverged; (2) removing all reference DNA profiles that have≥5% missing data; (3) rank all reference DNA profiles by identifying each reference DNA profile with a ranking value, wherein ranking value is determined based on the number of related reference DNA profiles in the full set of reference DNA profiles that is ranked from least to most and ties are broken by the number of ancestry-diverged reference DNA profiles in the full set of reference DNA profiles as ranked from most to least; and iteratively through the ranked reference DNA profiles, for each reference DNA profile: (i) if the reference DNA profile is not yet in a related sample set, add it to an unrelated sample set and add all related reference DNA profiles to the related sample set, and (ii) if the reference DNA profile is already in the related sample set, then skip to the next reference DNA profile, and repeat beginning at step (3)(i).
30. The method of any one of embodiments 1-5 and 7-29, wherein the one or more reference DNA profiles comprises at or about or at least or at least about 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles.
31. The method of any one of embodiments 1-5 and 7-29, wherein the one or more reference DNA profiles comprises at or about or at least or at least about 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles.
32. A nucleic acid library constructed using the method of any one of embodiments 6-31.
33. A plurality of primers that specifically hybridize to a plurality of target sequences comprising at least between at or about 5,000 to 50,000 single nucleotide polymorphisms (SNPs) in a nucleic acid sample, wherein amplifying the nucleic acid sample using the plurality of primers in one or more multiplex PCR reactions results in amplification products.
34. The plurality of primers of embodiment 33, wherein the nucleic acid sample comprises genomic DNA.
35. The plurality of primers of embodiment 33 or embodiment 34, wherein the nucleic acid sample comprises one or more enzyme inhibitors.
36. The plurality of primers of embodiment 35, wherein the one or more enzyme inhibitors comprise one or more inhibitors selected from the group consisting of hematin, heme, humic acid, indigo, and tannic acid.
37. The plurality of primers of any one of embodiments 23-36, wherein the nucleic acid sample comprises low-quality nucleic acid molecules and/or low quantity nucleic acid molecules.
38. The plurality of primers of embodiment 37, wherein the low quality nucleic acid molecules are degraded genomic DNA and/or fragmented genomic DNA.
39. The plurality of primers of embodiment 37 or embodiment 38, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.
40. The plurality of primers of any one of embodiments 37-39, wherein the low quality nucleic acid molecules have a DI of at least 1 and up to or less than 158.3.
41. The plurality of primers of any one of embodiments 33-36, wherein the nucleic acid sample comprises high quality nucleic acid molecules.
42. The plurality of primers of embodiment 41, wherein the high quality nucleic acid molecules have a DI of less than 1.
43. The plurality of primers of any one of embodiments 33-42, wherein the nucleic acid sample is a forensic sample.
44. The plurality of primers of any one of embodiments 33-43, wherein the nucleic acid sample is derived from a buccal swab, paper, fabric, or other substrate that is impregnated with saliva, blood, or other bodily fluid.
45. The plurality of primers of any one of embodiments 33-44, wherein the nucleic acid sample comprises between or between about 50 μg and 100 ng of genomic DNA.
46. The plurality of primers of any one of embodiments 33-45, wherein the nucleic acid sample comprises between or between about 100 pg and 5 ng of genomic DNA or between or between about 50 pg and 5 ng of genomic DNA.
47. The plurality of primers of embodiment 45 or embodiment 46, wherein the nucleic acid sample comprises at or about 1 ng of genomic DNA.
48. The plurality of primers of any one of embodiments 33-47, wherein the plurality of SNPs comprises kinship SNPs (kiSNPs).
49. The plurality of primers of any one of embodiments 33-48, wherein the plurality of SNPs comprises kiSNPs, biogeographical ancestry SNPs (aiSNPs), identity SNPs (iiSNPs), phenotype SNPs (piSNPs), x-chromosome SNPs (xSNPs), and y-chromosome SNPs (ySNPs).
50. The plurality of primers of any one of embodiments 33-48, wherein the plurality of SNPs comprises SNPs selected from one or more of the groups consisting of kiSNPs, aiSNPs, iiSNPs, piSNPs, xSNPs, and ySNPs.
51. The plurality of primers of any one of embodiments 48-50, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.
52. A method for constructing a DNA profile, comprising:
   providing a nucleic acid sample,
   amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions,
   sequencing the amplification products,
   determining the genotypes of the plurality of SNPs, thereby generating a DNA profile.
53. The method of embodiment 52, wherein the sequencing does not comprise whole genome sequencing (WGS).
54. The method of embodiment 52 or embodiment 53, wherein the nucleic acid sample comprises genomic DNA.
55. The method of any one of embodiments 52-54, wherein the nucleic acid sample comprises one or more enzyme inhibitors.
56. The method of embodiment 55, wherein the one or more enzyme inhibitors comprise one or more inhibitors selected from the group consisting of hematin, heme, humic acid, indigo, and tannic acid.
57. The method of any one of embodiments 52-56, wherein the nucleic acid sample comprises low-quality nucleic acid molecules and/or low quantity nucleic acid molecules.
58. The method of embodiment 57, wherein the low quality nucleic acid molecules are degraded genomic DNA and/or fragmented genomic DNA.
59. The method of embodiment 57 or embodiment 58, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.
60. The method of any one of embodiments 57-59, wherein the low quality nucleic acid molecules have a DI of at least 1 and up to or less than 158.3.
61. The method of any one of embodiments 52-56, wherein the nucleic acid sample comprises high quality nucleic acid molecules.
62. The method of embodiment 61, wherein the high quality nucleic acid molecules have a DI of less than 1.
63. The method of any one of embodiments 52-62, wherein the nucleic acid sample is a forensic sample.
64. The method of any one of embodiments 52-63, wherein the nucleic acid sample is derived from a buccal swab, paper, fabric, or other substrate that is impregnated with saliva, blood, or other bodily fluid.
65. The method of any one of embodiments 52-64, wherein the nucleic acid sample comprises between or between about 50 μg and 100 ng of genomic DNA.
66. The method of any one of embodiments 52-65, wherein the nucleic acid sample comprises between or between about 100 pg and 5 ng of genomic DNA or between or between about 50 pg and 5 ng of genomic DNA.
67. The method of embodiment 65 or embodiment 66, wherein the nucleic acid sample comprises at or about 1 ng of genomic DNA.
68. The method of any one of embodiments 52-67, wherein the plurality of SNPs comprises kinship SNPs.
69. The method of any one of embodiments 52-68, wherein the plurality of SNPs comprises kiSNPs, biogeographical ancestry SNPs (aiSNPs), identity SNPs (iiSNPs), phenotype SNPs (piSNPs), x-chromosome SNPs (xSNPs), and y-chromosome SNPs (ySNPs).
70. The method of any one of embodiments 52-69, wherein the plurality of SNPs comprises SNPs selected from one or more of the groups consisting of kiSNPs, aiSNPs, iiSNPs, piSNPs, xSNPs, and ySNPs.
71. The method of any one of embodiments 68-70, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.
72. A method of identifying genetic relatives of a DNA profile, comprising:
   calculating the degree of relationship of the DNA profile of any one of embodiments 52-71 to the one or more reference DNA profiles; and
   generating a family tree comprising the DNA profile in relation to the one or more reference DNA profiles.
73. The method of embodiment 72, further comprising generating a family tree comprising the DNA profile in relation to one or more DNA profiles.
74. The method of embodiment 72 or embodiment 73, wherein the one or more reference DNA profiles are part of a genealogy database.
75. The method of any one of embodiments 72-74, wherein the one or more reference DNA profiles comprises at or about or at least or at least about 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles.
76. The method of any one of embodiments 72-74, wherein the one or more reference DNA profiles comprises at or about or at least or at least about 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles.
77. A method of identifying genetic relatives of a DNA profile, comprising:
   calculating the degree of relationship of a DNA profile comprising genotypes of at least between at or about 5,000 to 50,000 SNPs to the one or more reference DNA profiles; and
   generating a family tree comprising the DNA profile in relation to the one or more reference DNA profiles.
78. The method of embodiment 77, wherein the DNA is generated by the method of any one of embodiments 52-71.
79. A kit comprising at least one container means, wherein the at least one container means comprises a plurality of primers of any one of embodiments 33-51.
80. The method of any one of embodiments 1-31, 51-71, and 77, wherein the plurality of SNPs comprises between about 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs.
81. The method of any one of embodiments 1-31, 51-71, and 77, wherein the plurality of SNPs comprises 10,230 SNPs.
82. The plurality of primers of any one of embodiments 33-51, wherein the plurality of SNPs comprises between about 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs.
83. The plurality of primers of any one of embodiments 33-51, wherein the plurality of SNPs comprises 10,230 SNPs.
84. The method of any one of embodiments 7-31, 52-71, and 77, further comprising generating a family tree comprising the DNA profile in relation to one or more DNA profiles.

### EXAMPLES

The following examples are included for illustrative purposes only and are not intended to limit the scope of the invention.

### Example 1: Generation of Sequence Libraries and Determination of Sensitivity

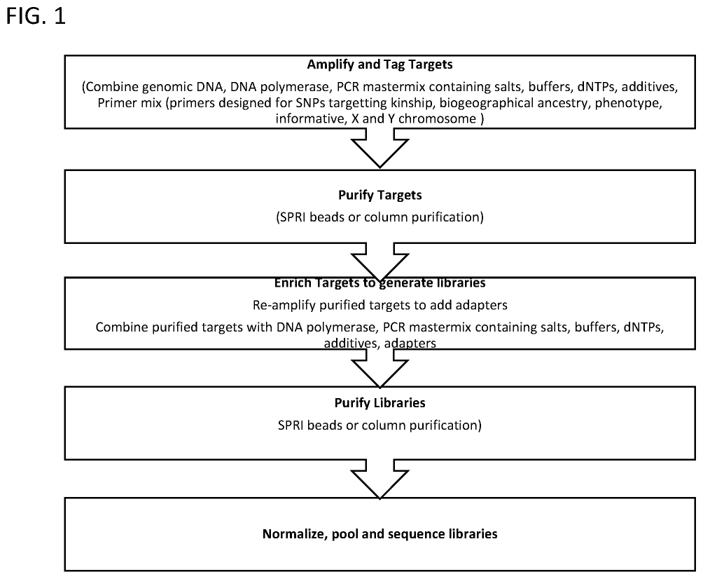

This Example describes a method of determining the sensitivity of the multiplex polymerase chain reaction described herein to generate libraries capable of being sequenced. FIG. 1 depicts an exemplary schematic of the method for generating a library capable of being sequenced described in this Example.

A. PCR Amplification of genomic DNA target

A multiplex polymerase chain reaction was performed to amplify 10,230 individual amplicons in a genomic DNA sample. Each primer pair was designed to selectively hybridize to, and promote amplification of a specific single nucleotide polymorphism (SNP) of the genomic DNA sample. A range of input genomic DNA was tested from 50 ng to 50 pg, more specifically, 5 ng, 2.5 ng, 1 ng, 500 pg, 250 μg, 100 pg and 50 pg). Briefly, 18.5 ml of a PCR mastermix containing sufficient buffer, dNTPs, MgCl2, salts and PCR additives such as glycerol was added to a single well of a 96-well PCR plate. 5 microliters of Primer Pool, containing 10,530 primer pairs, 2-4 Units of a DNA polymerase such as Phusion hot start DNA polymerase (Thermo Fisher, cat #F549L or any other thermostable DNA polymerase, 50 ng to 50 pg genomic DNA were also added.

The PCR plate was sealed and loaded into a thermal cycler (Veriti 96-well thermal cycler, Thermo Fisher Scientific, 4413964) and run on the temperate profile described below to generate the amplicon library.
98° C. for 3 minutes
18 cycles of:
96° C. for 45 seconds
80° C. for 10 seconds
54° C. for 4 minutes with applicable ramp mode
66° C. for 90 seconds with applicable ramp mode
68° C. for 10 minutes
Hold at 4° C.

After cycling, the amplicon library was held at 2-8° C. until proceeding to the purification step outlined below.

B. Purification of Amplicons from Input DNA and Primers

Two rounds of clean-up using MagBind Total Pure NGS beads (Omega Biotek, M1378-02) binding, wash, and elution at 1.6× and 0.6× volume ratios were found to remove genomic DNA and unbound or excess primers. The amplification and purification step outlined herein produces amplicons of about 150-350 bp in length. Purified amplicons are then used in a second round of PCR to add adapters for sequencing.

C. Enrichment of Purified Amplicons to Generate Libraries Capable of being Sequenced

A second round of PCR amplification is performed by combining 25 ml of purified amplicons from step above with 5 ml of adapters provided in Forenseq Kintelligence kit (Verogen PN:V16000120) and 20 ml of KPCR2 mastermix provided in Forenseq Kintelligence kit (Verogen PN:V16000120) in a 96 well PCR plate. The PCR plate was sealed and loaded into a thermal cycler (Veriti 96-well thermal cycler, Thermo Fisher Scientific, 4413964) and run on the temperate profile described below to generate the amplicon library.
98° C. for 30 seconds
15 cycles of:
98° C. for 20 seconds
66° C. for 30 seconds
72° C. for 30 seconds
72° C. for 1 minute
Hold at 4° C.

The libraries were purified using MagBind Total Pure NGS beads (Omega Biotek, M1378-02) binding, wash, and elution at 1X. The purified libraries were quantitated, normalized, denatured and diluted as per instructions given in Forenseq Kintelligence kit User Guide (Verogen PN:V16000120, the contents of which are hereby incorporated by reference in their entirety).

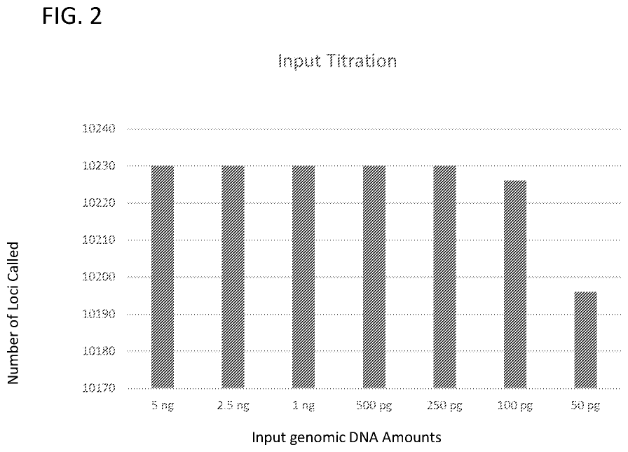

The denatured libraries were sequenced as per instructions on MiSeq FGx Sequencing System Reference Guide (document #VD2018006, the contents of which are hereby incorporated by reference in their entirety). As shown in FIG. 2, the number of loci detected were similar across a range of input genomic DNA titrations.

Results were analyzed using the Forenseq Universal Analysis Software 2.1 (Verogen, San Diego, CA) following the instructions outlined in Forenseq Universal Analysis Software 2.1, and provided in Reference Guide Document #VD2019002, the contents of which are hereby incorporated by reference in their entirety.

### Example 2: Generation of Sequence Libraries Using Degraded DNA

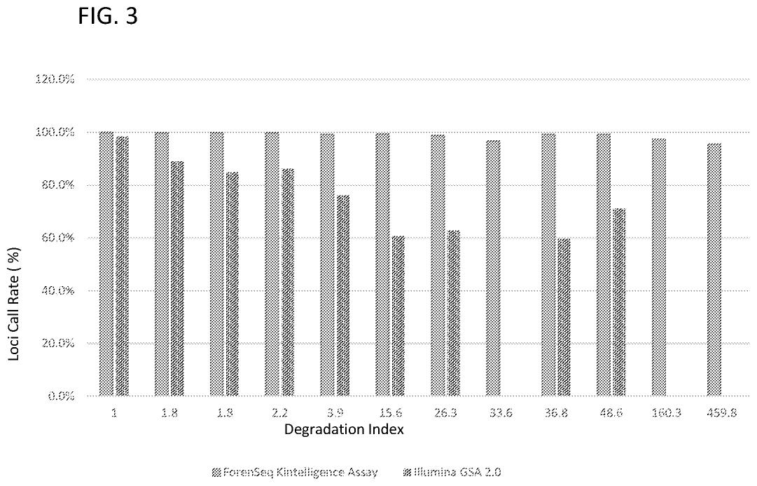

This Example describes the sequencing of DNA from low quantity and highly degraded samples. Degraded DNA A series of degraded blood DNA was obtained from Innogenomics (New Orleans, LA). The DNA samples were used to generate sequencing libraries as described in Example 1, with the exception that primer pairs for 10,327 loci were used in this example. The percentage of Loci detected (call rate) with degraded DNA using the assay described herein compared to Microarray (GSA) call rate is shown in FIG. 3. The degradation Index (DI) is shown on x-axis and the number of detected loci on Y-axis. These results show that even with highly degraded DNA with a DI of 158.3, the assay detected 9167 loci, which is sufficient to upload to the genealogy database to search for relatives. The alternative technologies such as Microarrays failed to detect any loci in samples with high degradation index.

### Example 3: Assessment of Activity of Inhibitors on Library Preparation

This Example describes assessment of the effect of PCR inhibitors on the preparation of libraries disclosed herein. DNA samples from crime scenes often contain co-purified impurities which inhibit PCR. PCR inhibition is the most common cause of PCR failure when adequate copies of DNA are present. Humic compounds, a series of substances produced during decay process have been considered as the materials contaminating DNA in soil, natural waters and recent sediments. Other common inhibitors include hematin (from blood), indigo (from blue jeans) and tannic acid.

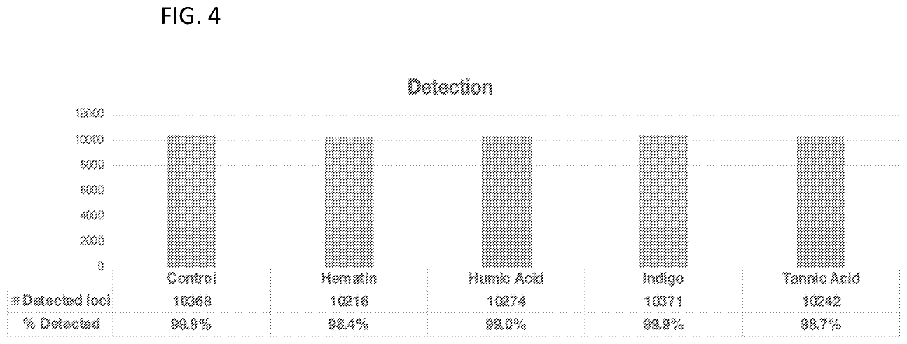

To assess the impact of inhibitors commonly found in forensic samples, library preparation was performed as described in Example 1, with the exceptions of 200 uM Hematin, 50 ng/uL Humic Acid, 133 uM Indigo, 16 uM Tannic Acid were spiked into the “Amplify and Tag targets” step above and primer pairs for 10380 loci were used. Results are shown in FIG. 4, with a PCR reaction without any inhibitors is labeled as Control.

### Example 4: Determination of Degree of Relationship

This Example describes exemplary results from samples prepared generally as described in Example 1 above.

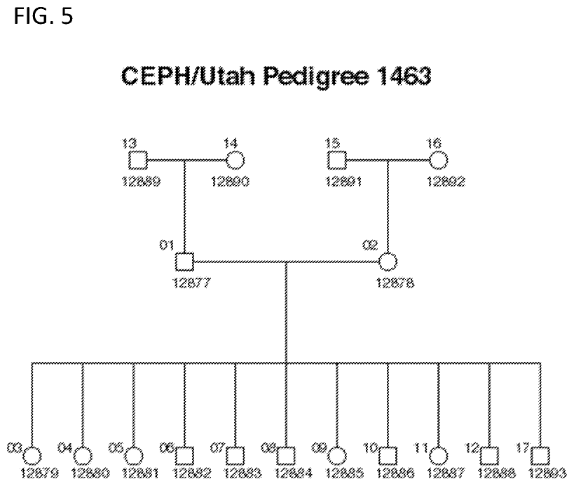

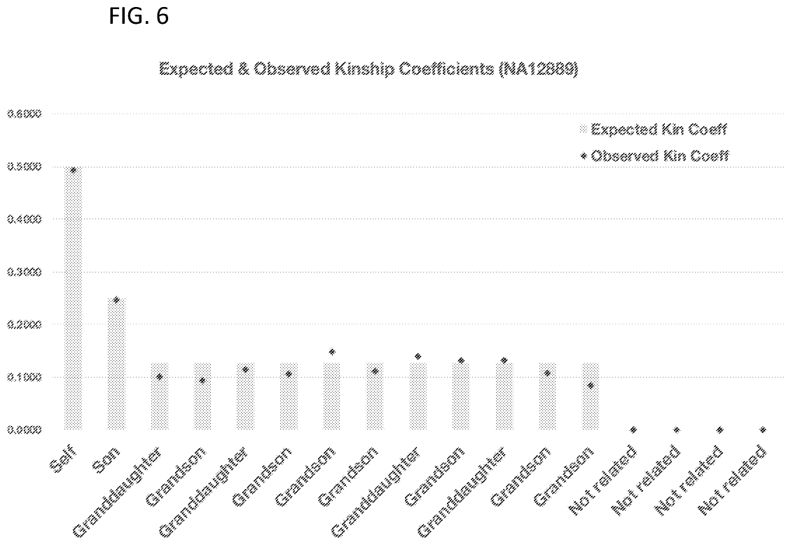

Illumina Global Screening Array (GSA) 2.0 were run with 200 ng each of 17 samples of Utah CEPH family 1463 DNA (Coriell Institute). The SNP calls were uploaded to the GEDmatch database (Verogen). An exemplary family tree is shown in FIG. 5. One of the samples, NA12889 (paternal grandfather) was run in the library preparation protocol as described in Example 1, run on ForenSeq UAS 2.1 module. The generated report was uploaded to the database and searched using the 1:many tool for searching relationships. The kinship coefficients from the algorithm in the database were compared to the expected kinship coefficients. The expected and observed kinship coefficients are shown in FIG. 6.

### Example 5: Kinship Coefficient Determination in Exemplary Case Study

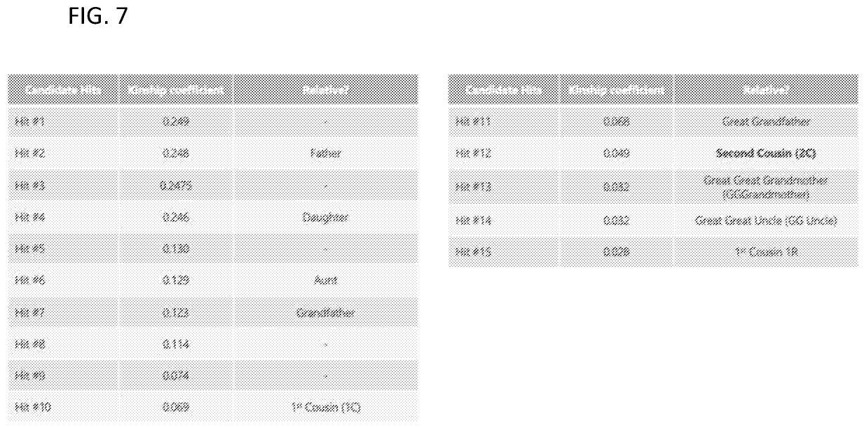

This Example describes the results of an exemplary case study using a sample SNP profile to determine kinship coefficient. The ability of the 1:many search algorithm to detect potential relatives was tested using 10 established pedigrees with 12-28 family members in the GEDmatch database. The sample SNP profile from the assay disclosed herein was considered to be of Mr. X=POI (Person of interest/unknown crime scene profile). Candidate hits, kinship coefficient and relative status are shown in FIG. 7.

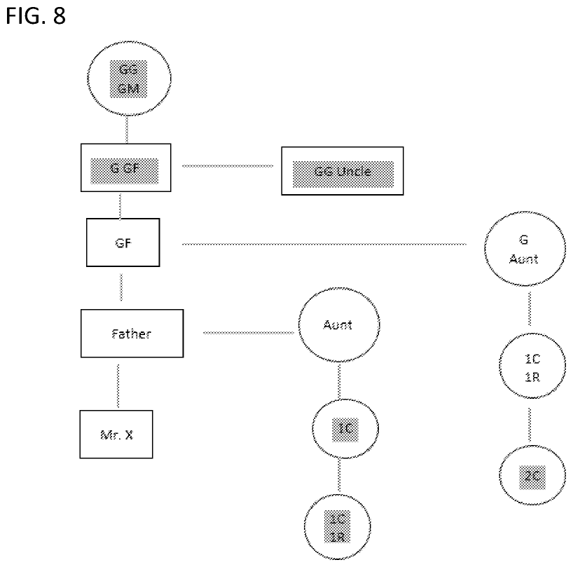

The results generated from the search algorithm were then used to generate the family tree for Mr. X as shown in FIG. 8. As shown in the family tree, Mr. X's first cousin (1C) and great grandfather (G GF) which are 3′ degree relationships; were returned within the first 11 candidate hits. Mr. X's Great Great Grandmother (GG GM), Great Great uncle (GG uncle) and First cousin once removed (1C1R), which are 4^{th} degree relationships were returned within the first 15 candidate hits. Mr. X's second cousin (2C), a 5^{th} degree relationship was the 12^{th} hit.

### Example 6: Generation of Sequence Libraries and Determination of Sensitivity, Including Assessment by the Type of Loci

This Example involves a method of determining the sensitivity of the multiplex polymerase chain reaction described herein to generate libraries capable of being sequenced, and includes an assessment by the type of loci.

Sequence libraries (sequenced nucleic acid libraries), also referred to as DNA profiles, were generated in the same manner as described in Example 1, except that results were analyzed using the Forenseq Universal Analysis Software version 2.2.

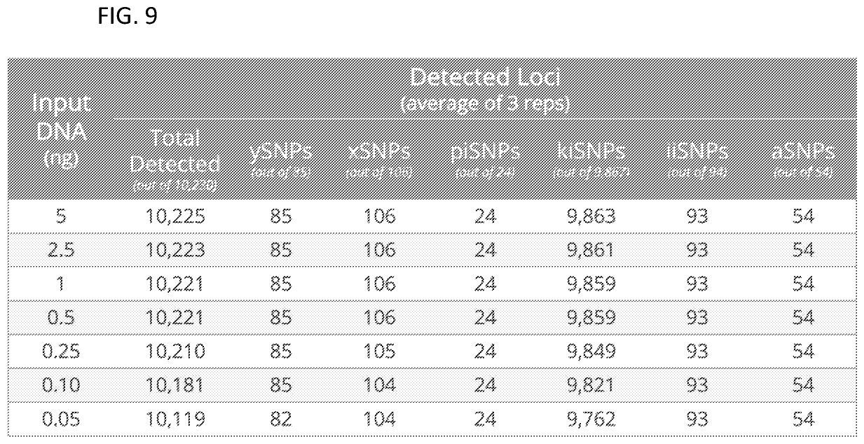

The results are shown in FIG. 9, which is a table summarizing the number of detected loci (as an average of three replicates) based on the amount of input DNA (ng) for each of the different types of loci, e.g., y-chromosome SNPs (ySNPs), x-chromosome SNPs (xSNPs), phenotype SNPs (piSNPs), kinship SNPs (kiSNPs), identity SNPs (iiSNPs), and biogeographical ancestry SNPs (aiSNPs), out of a total of 10,230 total loci being analyzed. Input titrations of genomic DNA tested included 5 ng, 2.5 ng, 1 ng, 0.5 ng (500 pg), 0.25 ng (250 pg), 0.10 ng (100 pg), and 0.05 ng (50 pg) of input genomic DNA. As shown in FIG. 9, the total detected SNPs, each of the amounts of input DNA ranging from 0.05 ng to 5 ng resulted in at least 98.9% (10,117) of the loci being detected, and the amounts of input DNA of 0.10 ng and greater resulted in at least 99.5% (10,179) of the loci being detected.

This data demonstrates that more than 10,000 loci can be detected at a high efficiency and a high sensitivity using different types of SNPs and using amounts of input DNA ranging from 0.05 ng (50 pg) to 5 ng.

### Example 7: ASSESSMENT OF ACTIVITY OF INHIBITORS ON SEQUENCE LIBRARY Preparation, Including Assessment by the Type of Loci

This Example describes an assessment of the effect of certain inhibitors on the preparation of sequence libraries (sequenced nucleic acid libraries) also referred to as DNA profiles, disclosed herein, including by type of loci being detected and sequenced. DNA samples from crime scenes often contain co-purified impurities which inhibit amplification. Common inhibitors include Hematin, Humic Acid, and Indigo.

To assess the impact of inhibitors commonly found in forensic samples, library preparation was performed as described in Example 1, except that results were analyzed using the Forenseq Universal Analysis Software version 2.2, and an assessment of the impact of certain inhibitors on amplification was performed as described in Example 3, with the exception that the inhibitors tested were as follows: 200 μM Hematin, 100 μM Hematin, 50 ng/μL, Humic Acid, 25 ng/μL, Humic Acid, 16 μM Tannic Acid, 8 μM Tannic Acid, 133 μM Indigo, and 66.5 μM Indigo were included in the amplification step as described in Example 1, and primer pairs for 10230 loci were used. A positive control reaction without any inhibitor included was also performed. 1 ng of input DNA was used.

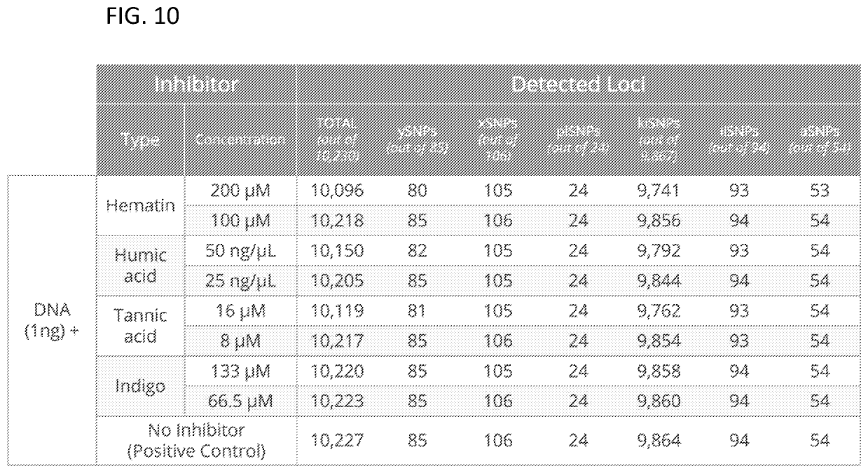

The results are shown in FIG. 10, which demonstrates that various SNPs including kiSNPs, ySNPs, xSNPs, piSNPs, iiSNPs, and aiSNPs can be amplified and detected in combination with one another in accordance with the methods described herein with a high rate of efficiency and detection, as demonstrated by, e.g., all or nearly all of the SNPs of each type being detected even when in the presence of the inhibitor. For instance, the number of detected kiSNPs, ySNPs, xSNPs, piSNPs, iiSNPs, and aiSNPs are each similar to the number detected in the positive control that lacked an inhibitor (FIG. 10). This data demonstrates that the presence of common inhibitors in samples does not have a detrimental impact on the ability to amplify more than 10,000 SNPs in PCR reactions using the methods described herein.

### Example 8: Assessment of Sequence Library Preparation Using DNA Samples Obtained Following a Mock Sexual Assault

This example describes the generation of sequence libraries (sequenced nucleic acid libraries) also referred to as DNA profiles, using DNA from mock sexual assault samples. Mock sexual assault DNA was obtained from samples collected at 9 hours and 22 hours after the occurrence of a mock sexual assault. DNA was isolated from the sperm fraction using a differential extraction method, with sperm fractions from both time points collected and saved for analysis. The amount of DNA from the sperm fraction that was available as input in the assay (for the generation of a sequence library) was only 500 pg, which is half of the recommended amount of 1 ng.

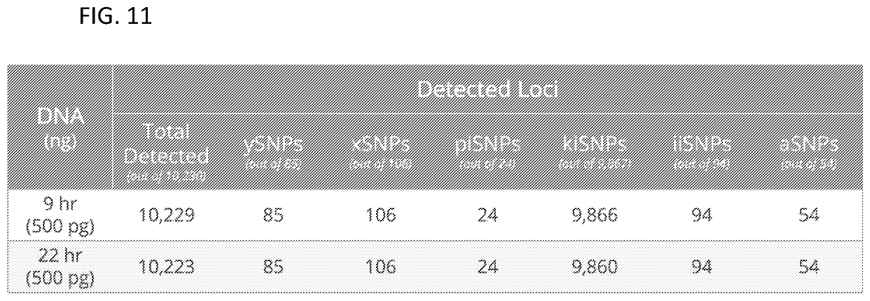

The DNA samples were used to generate sequence libraries (sequenced nucleic acid libraries) as described in Example 1, except that results were analyzed using the Forenseq Universal Analysis Software version 2.2. The percentage of loci detected (call rate) as well as the number of each type of SNP present in the assay are shown in FIG. 11. The results demonstrate that even with only 500 pg of input DNA, the majority of SNPs are detected, with 99.99% of all SNPs (10,229 out of 10,230 SNPs) being detected at the 9 hour time point, and 99.93% of all SNPs (10,223 out of 10,230 SNPs) being detected at the 22 hour time point. Specifically, all aiSNPs, iiSNPs, piSNPs, xSNPs, and ySNPs were detected at both the 9 hour and 22 hour time points after the occurrence of the mock sexual assault. Only one kiSNP out of 9,867 was not detected at the 9 hour time point, and only seven kiSNPs out of 9,867 were not detected at the 22 hour time point. The number of loci detected is sufficient to upload to the genealogy database to search for relatives.

This data demonstrates that the methods described herein can be used to detect more than 10,000 SNPs, including various kiSNPs, ySNPs, xSNPs, piSNPs, iiSNPs, and aiSNPs, and create sequence libraries, using only 500 pg of DNA at 9 hours and 22 hours after occurrence of a mock sexual assault, with more than 99.9% of all SNPs detected. Accordingly, the methods described herein are suitable for use in creating sequence libraries with less than recommended amounts of DNA, e.g., 500 pg, following criminal incidents, including sexual assaults.

### Example 9: Assessment of PCIA Carry-Over on Generation of Sequence Libraries from Saliva Samples

This Example describes the sequencing of nucleic acid libraries (e.g., to generate DNA profiles) from DNA derived from saliva samples that was extracted using organic extraction with the phenol-chloroform-isoamyl alcohol (PCIA) extraction method.

Saliva DNA was obtained from saliva samples where increasing amounts of the extraction reagent PCIA (e.g., no PCIA, light PCIA, moderate PCIA, and heavy PCIA) were intentionally left with the extracted DNA as carry-over, which simulates less than perfect extraction. PCIA, including its ingredient phenol, is a known inhibitor of PCR amplification.

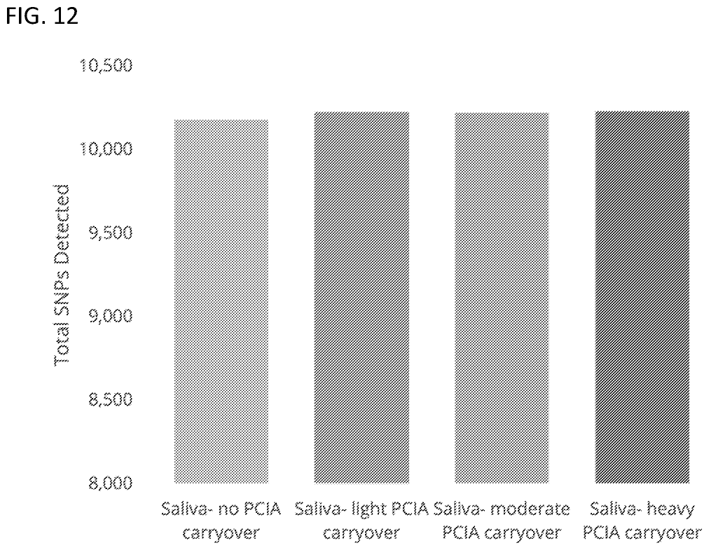

The DNA samples having no PCIA, light PCIA, moderate PCIA, or heavy PCIA were used to generate sequence libraries (sequenced nucleic acid libraries) as described in Example 1, except that results were analyzed using the Forenseq Universal Analysis Software version 2.2. The total number of SNPs detected for each sample was determined and is shown in FIG. 12. The results show that PCIA carry-over, even at high levels with heavy PCIA carry-over, does not affect the ability for the assay to detect SNPs since more than 10,170 SNPs were detected in each of the samples.

### Example 10: Assessment of Generation of Sequence Libraries from Blood Samples on Various Substrates and Impact of Heme

This example describes the sequencing of nucleic acid libraries (e.g., to generate DNA profiles) on DNA derived from blood samples deposited in different substrates typically found at crime scenes, including rust and denim, as well as a blood sample on a swab where only 420 pg of DNA was available, and blood samples extracted using CheleX™ where increasing levels of heme was carried over with the DNA. Heme is a known inhibitor of PCR amplification. Denim contains indigo dye, which is a known inhibitor of PCR amplification.

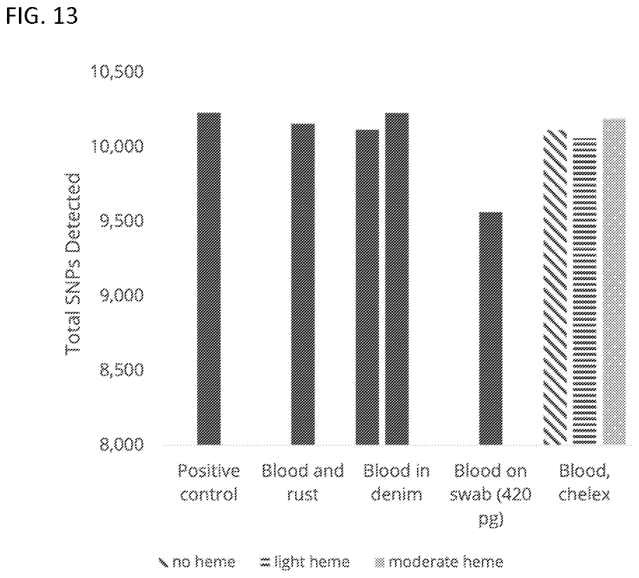

Each of the DNA samples was used to generate a sequence library (sequenced nucleic acid library) as described in Example 1, except that results were analyzed using the Forenseq Universal Analysis Software version 2.2, including a sample containing blood and rust, two blood samples in denim, a 420 pg blood sample on a swab, and blood samples with light or moderate amounts of heme carry-over or no heme as a control, as well as a positive control blood sample. The total number of SNPs detected for each sample and a reference control was determined and are shown in FIG. 13. The results show that the blood samples deposited in different substrates still allowed for the detection of 10,114 or more SNPs out of 10,230 total SNPs. The blood sample with only 420 pg yielded the detection of 9,563 SNPs, and the samples with heme yielded more than 10,000 SNPs detected, and the number of SNPs detected was not affected by the amount of heme present in the sample. This demonstrates that DNA extracted from blood samples deposited on various substrates commonly found at crime scenes can be used in accordance with the methods provided herein to detect more than 10,000 SNPs for forensic applications.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

What is claimed is:

1. A method for performing DNA-based kinship analysis, comprising:
providing a nucleic acid sample,
amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions, generating a nucleic acid library from the amplification products,
sequencing the nucleic acid library generated from the amplification products,
analyzing the sequences of the amplification products,
determining the genotypes of the plurality of SNPs, thereby generating a DNA profile, and
calculating the degree of relationship of the DNA profile to one or more reference DNA profiles.

2. A method for performing DNA-based kinship analysis, comprising:
providing a nucleic acid sample,
amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions,
generating a nucleic acid library from the amplification products
sequencing the nucleic acid library generated from the amplification products,
determining the genotypes of the plurality of SNPs, thereby generating a DNA profile, and
calculating the degree of relationship of the DNA profile to one or more reference DNA profiles.

3. The method of claim 1 or claim 2, wherein the sequencing is conducted using massively parallel sequencing (MPS).

4. The method of any one of claims 1-3, wherein the sequencing does not comprise whole genome sequencing (WGS).

5. The method of any one of claims 1-4, further comprising generating a family tree comprising the DNA profile in relation to one or more DNA profiles.

6. A method of constructing a nucleic acid library, comprising:
providing a nucleic acid sample,
amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating a nucleic acid library comprising amplification products, wherein the amplification is carried out in one or more multiplex PCR reactions.

7. The method of any one of claims 1-6, wherein the nucleic acid sample comprises genomic DNA.

8. The method of any one of claims 1-7, wherein the nucleic acid sample comprises one or more enzyme inhibitors.

9. The method of claim 8, wherein the one or more enzyme inhibitors comprise one or more inhibitors selected from the group consisting of hematin, heme, humic acid, indigo, and tannic acid.

10. The method of any one of claims 1-9, wherein the nucleic acid sample comprises low-quality nucleic acid molecules and/or low quantity nucleic acid molecules.

11. The method of claim 10, wherein the low quality nucleic acid molecules are degraded genomic DNA and/or fragmented genomic DNA.

12. The method of claim 10 or claim 11, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.

13. The method of claim 10 or claim 11, wherein the low quality nucleic acid molecules have a DI of at least 1 and up to or less than 158.3.

14. The method of any one of claims 1-9, wherein the nucleic acid sample comprises high quality nucleic acid molecules.

15. The method of claim 14, wherein the high quality nucleic acid molecules have a DI of less than 1.

16. The method of any one of claims 1-15, wherein the nucleic acid sample is a forensic sample.

17. The method of any one of claims 1-16, wherein the nucleic acid sample is derived from saliva, blood, semen, hair, teeth, or bone.

18. The method of claim 17, wherein the nucleic acid sample is derived from saliva, blood, or semen.

19. The method of any one of claims 1-16, wherein the nucleic acid sample is derived from a buccal swab, paper, fabric, or other substrate that is impregnated with saliva, blood, semen, or other bodily fluid.

20. The method of any one of claims 1-19, wherein the nucleic acid sample comprises between or between about 50 μg and 100 ng of genomic DNA.

21. The method of any one of claims 1-20, wherein the nucleic acid sample comprises between or between about 100 pg and 5 ng of genomic DNA or between or between about 50 pg and 5 ng of genomic DNA.

22. The method of claim 20 or claim 21, wherein the nucleic acid sample comprises at or about 1 ng of genomic DNA.

23. The method of any one of claims 1-22, wherein the plurality of SNPs comprises kinship SNPs (kiSNPs).

24. The method of any one of claims 1-23, wherein the plurality of SNPs comprises kiSNPs, biogeographical ancestry SNPs (aiSNPs), identity SNPs (iiSNPs), phenotype SNPs (piSNPs), x-chromosome SNPs (xSNPs), and y-chromosome SNPs (ySNPs).

25. The method of any one of claims 1-23, wherein the plurality of SNPs comprises SNPs selected from one or more of the groups consisting of kiSNPs, aiSNPs, iiSNPs, piSNPs, xSNPs, and ySNPs.

26. The method of any one of claims 23-25, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

27. A method for calculating degree of relatedness, comprising:
obtaining a DNA profile comprising genotypes of at least between at or about 5,000 to 50,000 SNPs; and
calculating the degree of relationship of the DNA profile to one or more reference DNA profiles.

28. A method for calculating degree of relatedness, comprising:
generating a DNA profile comprising genotypes of at least between at or about 5,000 to 50,000 SNPs; and
calculating the degree of relationship of the DNA profile to one or more reference DNA profiles.

29. The method of any one of claims 1-28, wherein the calculating the degree of relationship comprises a large cohort method comprising the steps of: (1) performing a KING-Robust kinship estimation between all pairs of a sample set comprising the one or more reference DNA profiles, wherein parings with a kinship coefficient>0.01 are identified as related and parings with a kinship coefficient<−0.025 are identified as ancestry-diverged; (2) removing all reference DNA profiles that have≥5% missing data; (3) rank all reference DNA profiles by identifying each reference DNA profile with a ranking value, wherein ranking value is determined based on the number of related reference DNA profiles in the full set of reference DNA profiles that is ranked from least to most and ties are broken by the number of ancestry-diverged reference DNA profiles in the full set of reference DNA profiles as ranked from most to least; and iteratively through the ranked reference DNA profiles, for each reference DNA profile: (i) if the reference DNA profile is not yet in a related sample set, add it to an unrelated sample set and add all related reference DNA profiles to the related sample set, and (ii) if the reference DNA profile is already in the related sample set, then skip to the next reference DNA profile, and repeat beginning at step (3)(i).

30. The method of any one of claims 1-5 and 7-29, wherein the one or more reference DNA profiles comprises at or about or at least or at least about 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles.

31. The method of any one of claims 1-5 and 7-29, wherein the one or more reference DNA profiles comprises at or about or at least or at least about 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles.

32. A nucleic acid library constructed using the method of any one of claims 6-31.

33. A plurality of primers that specifically hybridize to a plurality of target sequences comprising at least between at or about 5,000 to 50,000 single nucleotide polymorphisms (SNPs) in a nucleic acid sample, wherein amplifying the nucleic acid sample using the plurality of primers in one or more multiplex PCR reactions results in amplification products.

34. The plurality of primers of claim 33, wherein the nucleic acid sample comprises genomic DNA.

35. The plurality of primers of claim 33 or claim 34, wherein the nucleic acid sample comprises one or more enzyme inhibitors.

36. The plurality of primers of claim 35, wherein the one or more enzyme inhibitors comprise one or more inhibitors selected from the group consisting of hematin, heme, humic acid, indigo, and tannic acid.

37. The plurality of primers of any one of claims 23-36, wherein the nucleic acid sample comprises low-quality nucleic acid molecules and/or low quantity nucleic acid molecules.

38. The plurality of primers of claim 37, wherein the low quality nucleic acid molecules are degraded genomic DNA and/or fragmented genomic DNA.

39. The plurality of primers of claim 37 or claim 38, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.

40. The plurality of primers of any one of claims 37-39, wherein the low quality nucleic acid molecules have a DI of at least 1 and up to or less than 158.3.

41. The plurality of primers of any one of claims 33-36, wherein the nucleic acid sample comprises high quality nucleic acid molecules.

42. The plurality of primers of claim 41, wherein the high quality nucleic acid molecules have a DI of less than 1.

43. The plurality of primers of any one of claims 33-42, wherein the nucleic acid sample is a forensic sample.

44. The plurality of primers of any one of claims 33-43, wherein the nucleic acid sample is derived from a buccal swab, paper, fabric, or other substrate that is impregnated with saliva, blood, or other bodily fluid.

45. The plurality of primers of any one of claims 33-44, wherein the nucleic acid sample comprises between or between about 50 μg and 100 ng of genomic DNA.

46. The plurality of primers of any one of claims 33-45, wherein the nucleic acid sample comprises between or between about 100 pg and 5 ng of genomic DNA or between or between about 50 pg and 5 ng of genomic DNA.

47. The plurality of primers of claim 45 or claim 46, wherein the nucleic acid sample comprises at or about 1 ng of genomic DNA.

48. The plurality of primers of any one of claims 33-47, wherein the plurality of SNPs comprises kinship SNPs (kiSNPs).

49. The plurality of primers of any one of claims 33-48, wherein the plurality of SNPs comprises kiSNPs, biogeographical ancestry SNPs (aiSNPs), identity SNPs (iiSNPs), phenotype SNPs (piSNPs), x-chromosome SNPs (xSNPs), and y-chromosome SNPs (ySNPs).

50. The plurality of primers of any one of claims 33-48, wherein the plurality of SNPs comprises SNPs selected from one or more of the groups consisting of kiSNPs, aiSNPs, iiSNPs, piSNPs, xSNPs, and ySNPs.

51. The plurality of primers of any one of claims 48-50, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

52. A method for constructing a DNA profile, comprising:
providing a nucleic acid sample,
amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions,
sequencing the amplification products,
determining the genotypes of the plurality of SNPs, thereby generating a DNA profile.

53. The method of claim 52, wherein the sequencing does not comprise whole genome sequencing (WGS).

54. The method of claim 52 or claim 53, wherein the nucleic acid sample comprises genomic DNA.

55. The method of any one of claims 52-54, wherein the nucleic acid sample comprises one or more enzyme inhibitors.

56. The method of claim 55, wherein the one or more enzyme inhibitors comprise one or more inhibitors selected from the group consisting of hematin, heme, humic acid, indigo, and tannic acid.

57. The method of any one of claims 52-56, wherein the nucleic acid sample comprises low-quality nucleic acid molecules and/or low quantity nucleic acid molecules.

58. The method of claim 57, wherein the low quality nucleic acid molecules are degraded genomic DNA and/or fragmented genomic DNA.

59. The method of claim 57 or claim 58, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.

60. The method of any one of claims 57-59, wherein the low quality nucleic acid molecules have a DI of at least 1 and up to or less than 158.3.

61. The method of any one of claims 52-56, wherein the nucleic acid sample comprises high quality nucleic acid molecules.

62. The method of claim 61, wherein the high quality nucleic acid molecules have a DI of less than 1.

63. The method of any one of claims 52-62, wherein the nucleic acid sample is a forensic sample.

64. The method of any one of claims 52-63, wherein the nucleic acid sample is derived from a buccal swab, paper, fabric, or other substrate that is impregnated with saliva, blood, or other bodily fluid.

65. The method of any one of claims 52-64, wherein the nucleic acid sample comprises between or between about 50 μg and 100 ng of genomic DNA.

66. The method of any one of claims 52-65, wherein the nucleic acid sample comprises between or between about 100 pg and 5 ng of genomic DNA or between or between about 50 pg and 5 ng of genomic DNA.

67. The method of claim 65 or claim 66, wherein the nucleic acid sample comprises at or about 1 ng of genomic DNA.

68. The method of any one of claims 52-67, wherein the plurality of SNPs comprises kinship SNPs.

69. The method of any one of claims 52-68, wherein the plurality of SNPs comprises kiSNPs, biogeographical ancestry SNPs (aiSNPs), identity SNPs (iiSNPs), phenotype SNPs (piSNPs), x-chromosome SNPs (xSNPs), and y-chromosome SNPs (ySNPs).

70. The method of any one of claims 52-69, wherein the plurality of SNPs comprises SNPs selected from one or more of the groups consisting of kiSNPs, aiSNPs, iiSNPs, piSNPs, xSNPs, and ySNPs.

71. The method of any one of claims 68-70, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

72. A method of identifying genetic relatives of a DNA profile, comprising:
calculating the degree of relationship of the DNA profile of any one of claims 52-71 to the one or more reference DNA profiles; and
generating a family tree comprising the DNA profile in relation to the one or more reference DNA profiles.

73. The method of claim 72, further comprising generating a family tree comprising the DNA profile in relation to one or more DNA profiles.

74. The method of claim 72 or claim 73, wherein the one or more reference DNA profiles are part of a genealogy database.

75. The method of any one of claims 72-74, wherein the one or more reference DNA profiles comprises at or about or at least or at least about 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles.

76. The method of any one of claims 72-74, wherein the one or more reference DNA profiles comprises at or about or at least or at least about 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 reference DNA profiles.

77. A method of identifying genetic relatives of a DNA profile, comprising:
calculating the degree of relationship of a DNA profile comprising genotypes of at least between at or about 5,000 to 50,000 SNPs to the one or more reference DNA profiles; and
generating a family tree comprising the DNA profile in relation to the one or more reference DNA profiles.

78. The method of claim 77, wherein the DNA is generated by the method of any one of claims 52-71.

79. A kit comprising at least one container means, wherein the at least one container means comprises a plurality of primers of any one of claims 33-51.

80. The method of any one of claims 1-31, 51-71, and 77, wherein the plurality of SNPs comprises between about 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs.

81. The method of any one of claims 1-31, 51-71, and 77, wherein the plurality of SNPs comprises 10,230 SNPs.

82. The plurality of primers of any one of claims 33-51, wherein the plurality of SNPs comprises between about 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs.

83. The plurality of primers of any one of claims 33-51, wherein the plurality of SNPs comprises 10,230 SNPs.

84. The method of any one of claims 7-31, 52-71, and 77, further comprising generating a family tree comprising the DNA profile in relation to one or more DNA profiles.
